(19) United States
(12) Patent Application Publication
(10) Pub. No.: US 2013/0316397 A1
(43) Pub. Date: Nov. 28, 2013

(54) Cell-Free Polypeptide Synthesis

(71) Applicant: The Board of Trustees of the Leland Standford Junior University, Palo Alto, CA (US)

(72) Inventors: Isoken Airen, Schaumburg, IL (US); James Robert Swartz, Menlo Park, CA (US)

(21) Appl. No.: 13/871,773

(22) Filed: Apr. 26, 2013

Related U.S. Application Data
(60) Provisional application No. 61/640,563, filed on Apr. 30, 2012.

(51) Int. Cl. C12P 21/00; C12N 15/10

(52) U.S. Cl. CPC C12P 21/00; C12N 15/1068
USPC 435/68.1; 435/194; 506/9

(57) ABSTRACT

Methods, microbial strains and reaction mixtures for cell-free synthesis of polypeptides are provided. The methods of the invention utilize a reaction mixture comprising microbial cell extracts that are modified in the protein component relative to an extract from a native cell. The modification may be one or both of (i) increased levels of proteins that increase synthetic yield; and (ii) decreased levels of proteins that decrease synthetic yield. The modification may result from a genetic modification of the microbial cell, or from ex vivo supplementation or depletion of an extract.

### FEDERALLY SPONSORED RESEARCH AND DEVELOPMENT

This invention was made with Government support under contract Al057229 awarded by the National Institutes of Health.

### BACKGROUND OF THE INVENTION

Escherichia coli is a widely used organism for the expression of heterologous proteins. It easily grows to a high cell density on inexpensive substrates to provide excellent volumetric and economic productivities. Well established genetic techniques and various expression vectors further justify the use of Escherichia coli as a production host.

Over the past decade, the productivity of cell-free systems has improved by several orders of magnitude, making cell free protein synthesis a practical technique for laboratory-scale research and provides a platform technology for high-throughput protein expression. Cell-free technologies are also finding use as an alternative means to the in vivo large-scale production of protein pharmaceuticals.

Cell-free protein synthesis offers several advantages over conventional, in vivo, protein expression methods. Cell-free systems can direct most, if not all, of the metabolic resources of the cell towards the exclusive production of one protein. Moreover, the lack of a cell wall in vitro is advantageous since it allows for better control of the synthesis environment. For example, tRNA levels can be changed to reflect the codon usage of genes being expressed. Also, the redox potential, pH, or ionic strength can be altered with greater flexibility than in vivo since we are not concerned about cell growth or viability. Furthermore, direct recovery of purified, properly folded protein products can be easily achieved.

In vitro translation is also recognized for its ability to incorporate unnatural and isotope-labeled amino acids as well as its capability to produce proteins that are unstable, insoluble, or cytotoxic in vivo. In addition, cell-free protein synthesis may play a role in revolutionizing protein engineering and proteomic screening technologies. The cell-free method bypasses the laborious processes required for cloning and transforming cells for the expression of new gene products in vivo and is becoming a platform technology for this field.

Methods of increasing the yield of product from cell-free protein synthesis is of considerable interest for research and production. The present invention addresses these issues.

### PUBLICATIONS

Patent documents relating to in vitro protein synthesis include US patents and published applications: U.S. Pat. No. 6,168,931; U.S. Pat. No. 6,994,986; U.S. Pat. No. 6,337,191; U.S. Pat. No. 6,548,276; U.S. Pat. No. 7,041,479; U.S. Pat. No. 7,338,789; U.S. Pat. No. 7,341,852; US-2005-0054044; US 2007-0154983; US 2009-0042244; U.S. Pat. No. 7,312,049; US 2009-0029414; US 2009-0317861; U.S. Pat. No. 7,871,794. A bacterial strain useful for cell-free protein synthesis is described by Calhoun and Swartz, Total amino acid stabilization during cell-free protein synthesis reactions. J. Biotechnol. 2006, 123, 193-203.

### SUMMARY OF THE INVENTION

Methods, microbial strains and reaction mixtures for cell-free synthesis of polypeptides are provided. The methods of the invention utilize a reaction mixture comprising microbial cell extracts, which provide biological materials such as ribosomes, protein factors and the like that are necessary for efficient synthesis. The methods of the invention utilize a reaction mixture comprising microbial cell extracts that are modified in the protein component relative to an extract from a native cell. The modification may be one or both of (i) increased levels of proteins that increase synthetic yield; and (ii) decreased levels of proteins that decrease synthetic yield. The modification may result from a genetic modification of the microbial cell, or from ex vivo supplementation or depletion of an extract.

In some embodiments of the invention, a microbial cell extract is modified in the protein content relative to a native cell by comprising increased levels of expression of a gene identified as a positive effecter in any one of Tables 6A, 6B and 7. In other embodiments, a microbial cell extract is modified in the protein content relative to a native cell by decreased levels of a gene identified as a negative effecter in any one of Tables 8A, 6B and 7. Genes of interest as positive and negative effecters include, without limitation those categorized as follows: (1) Factors affecting the energy (ATP and GTP) supply (for transcription, translation, and protein folding processes); (2) Factors affecting the supply of nucleotide (NTPs) and amino acids for polymerization reactions (i) Nucleotide (NTP) supply (for energy and transcription); (ii) Amino acid supply (for translation); (3) Factors affecting transcription; (4) Factors affecting RNA stability; (5) Factors affecting translation; (6) Factors affecting both transcription and translation; (7) Factors affecting protein folding; (8) Factors affecting protein stability; and (9) Factors with unexplained influences. In some embodiments, individual effectors are validated by the methods described herein, e.g. by adding purified positive effectors to a candidate cell free polypeptide synthesis reaction; or by deletion of a coding sequence for a negative effector, and comparing the yield of one or more polypeptides with a control synthetic reaction.

Increased product yield relative is obtained by performing synthesis with a reaction mixture that is supplemented with proteins shown herein to increase yield of product. Such supplementing proteins may include one or more of bacterial AckA, EF-Tu, HchA, IbpA/B and IF 1-3. In some embodiments the supplementing protein is AckA. In some embodiments the supplementing proteins are AckA and one or more of EF-Tu, HchA, IbpA/B and IF 1-3. In some embodiments the supplementing proteins are all of AckA, EF-Tu, HchA, IbpA/B and IF 1-3. The proteins may be endogenously produced in the microbial cell from which the extracts for polypeptide synthesis are obtained, e.g. by genetically modifying the microbial cell for overproduction of the protein(s). In some embodiments, a microbial strain is provided, in which said genetic modifications are present, to overproduce the supplementing proteins of the invention. The supplementing proteins can alternatively be produced in the cell-free reaction system during production, or are exogenously produced and added to the reaction mixture. In reaction mixtures where AckA is supplemented, the reaction mixture may require pH buffers to offset an increase in acetic acid concentration caused by the AckA-catalyzed energy regeneration.

In related embodiments, methods, microbial strains, and reaction mixtures for cell-free synthesis of polypeptides are provided, where the reaction mixture comprises microbial cell extracts in which the extracts are derived from genetically modified microbial strains that are deficient in the enzyme ribonuclease II (rnb), where the gene encoding rnb may be deleted or otherwise inactivated.

In some embodiments, the microbial strain, extracts derived therefrom and reaction mixtures comprising such extracts combine (i) a deficiency in rnb and (ii) supplementing proteins including one or more of bacterial AckA, EF-Tu, HchA, IbpA/B and IF 1-3. Production of polypeptides in a cell-free synthesis reaction deficient in rnb and supplemented with AckA, EF-Tu, HchA, IbpA/B and IF 1-3 provide for increased polypeptide synthesis relative to a control system, e.g. where rnb is present and AckA, EF-Tu, HchA, IbpA/B and IF 1-3 are present at native levels. The increase in polypeptide product can be 100%, 200%, 300% or more.

Reaction mixes are optionally supplemented during the course of the reaction with substrate monomers that are limiting for the polypeptide of interest for production. In particular, the specific amino acid composition of the polypeptide being produced can result in a selective depletion of over-represented amino acids. Supplementing monomers include one, two, three, four or more amino acids; and may include one or more nucleotides or ribonucleotides. The determination of whether a substrate monomer is limiting for a polypeptide of interest may be determined empirically by performing test reactions and analyzing the amino acid pool following synthesis, by test supplementation, etc. Alternatively an analysis of the amino acid composition of a polypeptide may be performed and used to predict which substrate monomers will be limiting.

In some embodiments of the invention, a method of enhancing synthesis of a protein in a cell free protein synthesis (CFPS) reaction is provided. The methods survey the genome of an organism that is a source for biological extracts used in the CFPS by sequential expression to identify candidate effector genes, and described in Example 1. Combinations of genes from different metabolic groups, including without limitation: factors affecting the energy (ATP and GTP) supply (for transcription, translation, and protein folding processes); factors affecting the supply of nucleotide (NTPs) and amino acids for polymerization reactions (i) Nucleotide (NTP) supply (for energy and transcription); (ii) amino acid supply (for translation); factors affecting transcription; factors affecting RNA stability; factors affecting translation; factors affecting both transcription and translation; factors affecting protein folding; factors affecting protein stability; and factors with unexplained influences are used, where the candidate effector gene can be deleted from the biological extract, synthesized exogenously and added to the reaction mix, or expressed in the reaction mix itself. The reaction mix modified by the candidate effector genes is used to express a test, or target gene. Various combinations are tested, and those providing for enhanced synthesis are then further tested by adjusting the substrate concentration, pH, iconicity, etc. to determine the conditions that fully activate the catalytic power of the reaction mixture.

### BRIEF DESCRIPTION OF THE DRAWINGS

FIG. 1: Effect of YhbV enrichment on protein stability in the CFPS system. Total protein degradation was observed in CFPS reaction mixtures that were enriched for the predicted protease YhbV (A), an observation that is consistent with the homology-based assigned protein function. Such extensive proteolysis did not occur in the standard CFPS system (B).

FIG. 2: Effect of predicted endonucleases on plasmid DNA stability in CFPS reaction mixtures. The degradation pattern of plasmid DNA template, pY71L.GFP (2.45 kb), was studied in the presence of the following predicted endonucleases in the CFPS reaction mixture, without the addition of the S30 cell extract or NTPs: YhbQ (lane 1); YihG (lane 2); and YhaV (lane 3). The digestion reaction was incubated for 2 hours at 37° C., and the entire reaction was loaded on a 1×TBE, 1.0% (w/v) ethidium bromide-stained agarose gel. Various degrees of plasmid DNA digestion were caused by the different endonucleases. These observations are consistent with the predicted protein functions. Markers (lane M) correspond to the following band sizes (in base pairs, from top to bottom): 0.25, 0.5, 0.75, 1, 1.5, 2, 2.5, 3, 4, 5, 6, 8 and 10 kb (eENZYME, Montgomery Village, Md., USA). The position of uncut plasmid (lane 4) is shown by the arrow.

FIG. 3: Effect of glycerol kinase (GlpK) enrichment on the organic acid and nucleotide composition in the CFPS system. (A) The concentration of glycerol was reduced from baseline (black [-] line) when GlpK was expressed in the first-round of protein synthesis (gray [ [symbol] ] line), (n=3). (B) After the first-round expression of GlpK (gray [ [symbol] ] line), the concentration of ATP was reduced to barely detectable levels from a baseline value (black [ [symbol] ] line). This depletion of ATP was presumably the cause of the complete inhibition of protein synthesis observed. (n=3)

FIG. 4: Stabilization of reaction pH in CFPS system supplemented with acetate kinase (AckA). (A) The concentration of acetic acid in the CFPS system increased by 50% with the supplementation of AckA. This increase was largely due to the AckA-catalyzed energy regeneration reaction, and consequently, caused a significant drop in the reaction pH. (n=3). (B) The pH of CFPS reaction mixtures dropped by ˜15% when AckA was supplemented into the system (solid gray [ [symbol] ] line: standard system without AckA; solid black [-] line: AckA-supplemented system). The reaction pH was stabilized with the addition of 90 mM Bis-Tris (pH 7.2) (dashed black [- -] line). (n=3)

FIG. 5: Modification of standard CFPS for enhanced productivity with re-optimization of the small molecule environment and addition of targeted biocatalysts. The stabilization of the pH of the AckA-supplemented CFPS system enhanced GFP yields by ˜35% over the observed plateau value of 2 mg/mL (AckA+pH control). (n=3). The replenishment of the CFPS reaction mixtures with the limiting small molecules (L-phenylalanine, L-leucine, L-threonine, L-glycine, and UTP) in a semi-fed-batch manner improved GFP yields by ˜25% over the observed plateau concentration (AckA+fed-batch). (n=3) When these changes were applied to the AckA-supplemented CFPS system, GFP accumulation was enhanced by ˜150% over the standard conditions (AckA+pH control+fed-batch). (n=3) The 8-effector combination (AckA, HchA, IbpA, IbpB, IF1, IF2, IF3, and EF-Tu) that was identified by the DoE analysis improved GFP production by ˜220% over the standard system, resulting in soluble yield of ˜3.5 mg/mL (effector combo+pH control+fed-batch). (n=3)

FIG. 6: Concentration profile of depleting amino acids in the AckA-supplemented CFPS system. The concentration levels of several amino acids significantly reduced during the incubation period, which was largely due to the high content of each amino acid in the translated protein, green fluorescent protein (GFP). It is possible that the concentrations fell below the Kₘ values of the respective synthetases. (n=3)

FIG. 7: Concentration profile of NTPs in the AckA-supplemented CFPS system. The supply of UTP depleted within 4 hrs of the incubation period, whereas the concentrations of the other NTPs were stable at ˜0.4-0.6 mM. (n=3)

FIG. 8: Effect of rnb deletion from standard CFPS extract source cell genome on stability of mRNA transcripts. In vitro transcribed-³H-labelled GFP mRNA transcripts were incubated in CFPS reaction mixtures, without the addition of T7 RNA polymerase and DNA template, using S30 cell extracts prepared from the standard and rnb-deletion cell strains, KC6 and KC6Δrnb, respectively. Reaction samples were quenched at various times during the incubation period to analyze the mRNA stability via TCA precipitation (A) and denaturing agarose gels [1.0% (w/v)](B, C). With the KC6Δrnb cell extract, the mRNA transcript was stable for up to 8 hrs in the CFPS system (A: solid [-] line; B), whereas the transcript had greatly degraded by t_{reaction}=3 hr in the standard system (A: dashed [- -] line; C). (n=3) Markers (lane M) correspond to the following band sizes (in base pairs, from top to bottom): 300, 400, 500, 600, 800, and 1000 bp (Norgen Biotek Corp, Thorold. ON, Canada).

FIG. 9: Effect of the deletion of genes encoding highly-negative effectors from cell extract source cell genome on cell-free protein expression. Four single-gene deletion strains were created, targeting the genes of highly-negative effectors, and were used to prepare cell extracts. Only the CFPS system using the KC6Δrnb-derived cell extract showed improved performance over that of the standard system. The total and soluble yields of GFP were enhanced by ˜70%. (n=3)

FIG. 10: Effect of rnb deletion from standard CFPS extract source cell genome on duration of cell-free protein accumulation. The period of protein accumulation was extended to 8 hrs in the CFPS system using the S30 cell extract prepared from the rnb-deletion cell strain, KC6Δrnb, while protein production in the reaction mixtures containing the standard cell extract from strain KC6 stops within 5 hrs of the incubation period. This extended period of protein synthesis was presumably due to the stabilization of the mRNA transcript. (n=3)

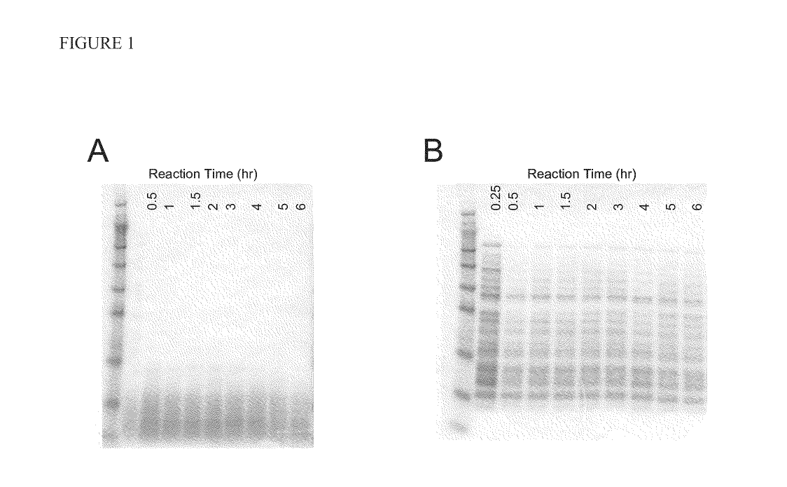

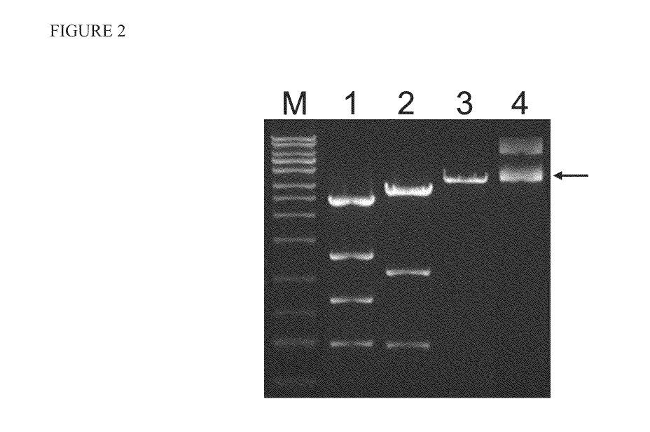

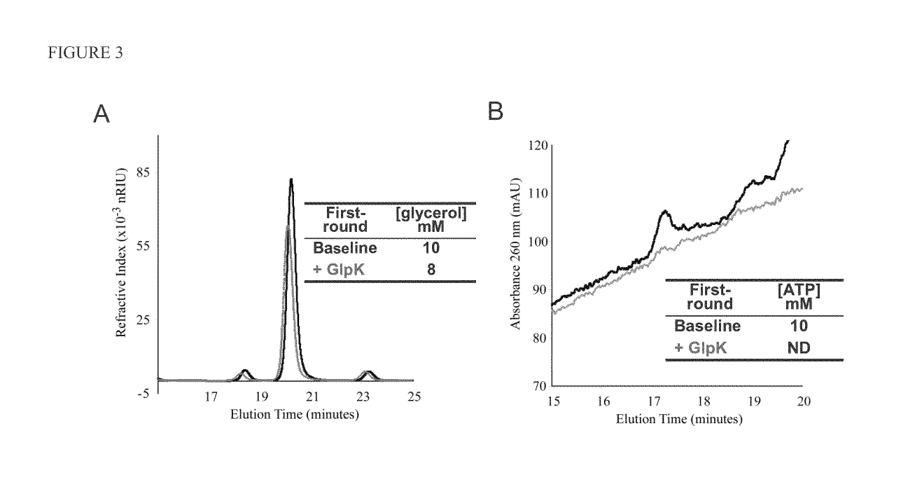

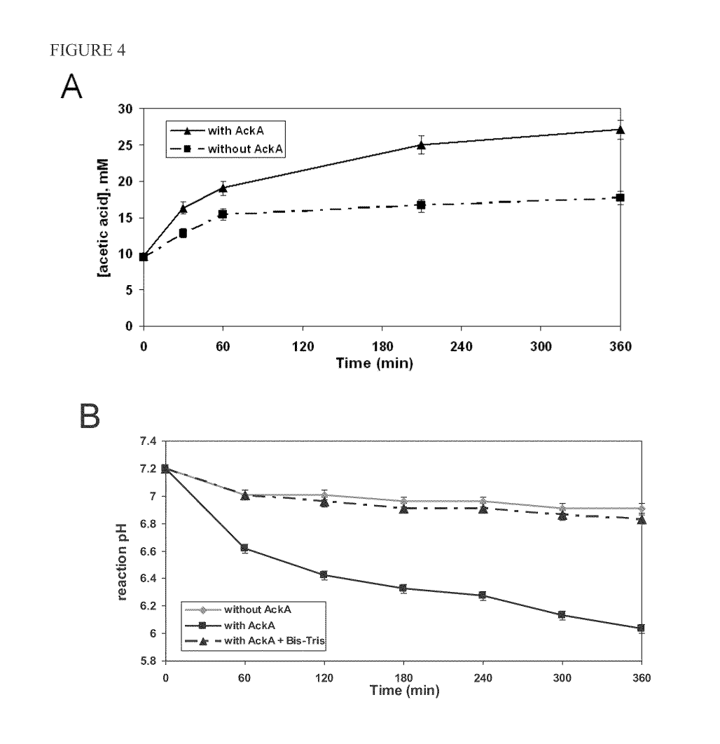

### DETAILED DESCRIPTION OF THE EMBODIMENTS

Methods, microbial strains and reaction mixtures for cell-free synthesis of polypeptides are provided. The methods of the invention utilize a reaction mixture comprising microbial cell extracts that are modified in the protein component relative to an extract from a native cell. The modification may be one or both of (i) increased levels of proteins that increase synthetic yield; and (ii) decreased levels of proteins that decrease synthetic yield. The modification may result from a genetic modification of the microbial cell, or from ex vive supplementation or depletion of an extract.

In one embodiment of the invention, a bacterial strain is provided in which genetic modifications have been made in enzymes affecting polypeptide synthesis. In some such strains, a negative effector from Table 6A, 6B or 7 is knocked out. In some such strains, the gene encoding rnb is “knocked out”, where synthesis of the targeted enzyme is substantially absent, through deletion of all or part of the coding sequence; deletion of all or part of the relevant promoter or operator sequence; introduction of one or more stop codons at a position in the coding sequence that will substantially ablate expression; and the like. The use of E. coli is of particular interest. In some such strains, a negative effector from Table 6A, 6B or 7 is knocked out.

A bacterial strain is also provided that is genetically modified by introduction of an expression construct that increases the expression of one or more of a positive effectors protein set forth in Table 6A, 6B or 7, which positive effectors may comprise without limitation one or more of bacterial proteins AckA, EF-Tu, HchA, IbpA/B and IF 1-3. In some embodiments the protein is AckA. In some embodiments the proteins are AckA and one or more of EF-Tu, HchA, IbpA/B and IF 1-3. In some embodiments the proteins are all of AckA, EF-Tu, HchA, IbpA/B and IF 1-3. In some embodiments the bacterial strain is further modified by a deficiency in rnb as described above.

In certain embodiments, any one of the genetically modified bacterial strains described herein is also modified by a knock-out of genes affecting amino acid synthesis, for example speA, tnaA, sdaA, adaB, and gshA. Additional genetic modifications may also be made to the microbial strain, for example the deletion of tonA and endA genes to protect against bacteriophage infection and stabilize DNA within the system. Such bacterial strains are described, for example in U.S. Pat. No. 7,312,049, and in Calhoun and Swartz (2006) J. Biotechnol. 123:193-203, both herein specifically incorporated by reference.

A cellular extract of a bacterial strain as described above is provided, which extract may be provided in a fresh or frozen form, and may further be formulated into a reaction mix suitable for polypeptide synthesis. Such extracts are obtained by any of the methods known in the art for the purpose of cell-free protein synthesis. In one example of such methods, cells are grown in media to the appropriate optical density, harvested by centrifugation and washed in S30 buffer (10 mM Tris, 8.2, 14 mM Mg acetate, 60 mM potassium acetate, 1 mM DTT). After the final wash, the cells are resuspended in S30 buffer and disrupted, e.g. with a French press. The lysate is then centrifuged, and the withdrawn supernatant used as the extract. The extract is optionally further purified by dialysis, centrifugation, dilution with appropriate salts, and the like. Methods for producing active extracts are known in the art, for example they may be found in Pratt (1984), coupled transcription-translation in prokaryotic cell-free systems, p. 179-209, in Hames, B. D. and Higgins, S. J. (ed.), Transcription and Translation: a practical approach, IRL Press, New York. Kudlicki et al. (1992) Anal Biochem 206(2):389-93 modify the S30 E. coli cell-free extract by collecting the ribosome fraction from the S30 by ultracentrifugation.

A cellular extract for the purpose of cell-free protein synthesis, including without limitation the genetically modified bacterial strains described herein, may be supplemented with one or more of bacterial proteins AckA, EF-Tu, HchA, IbpA/B and IF 1-3. In some embodiments the protein is AckA. In some embodiments the proteins are AckA and one or more of EF-Tu, HchA. IbpA/B and IF 1-3. In some embodiments the proteins are all of AckA, EF-Tu, HchA, IbpA/B and IF 1-3.

In another embodiment of the invention, methods of cell-free polypeptide synthesis are provided, where the reaction mixture comprises a cell extract as described above. These methods are applicable to continuous, semi-continuous and batch reactions. For some synthetic reactions, e.g. multiplexed reactions, it is preferable to use batch rather than a semi-continuous system. For batch synthesis methods, the reaction mix is optionally supplemented during the course of the reaction with substrate monomers that are limiting for the polypeptide of interest for production, e.g. by hourly, bihourly, etc. addition of monomers. Supplementing monomers include one, two, three, four or more amino acids; and may include one or more nucleotides or ribonucleotides. Each monomer may be added at a concentration of at least about 0.1 mM, at least about 0.25 mM, at least about 0.5 mM, and not more than about 2 mM.

In reaction mixtures where AckA is supplemented, the reaction mixture may require pH buffers to offset an increase in acetic acid concentration caused by the AckA-catalyzed energy regeneration, where the buffer may be provided at a final concentration of at least 25 mM, at least 50 mM, at least 75 mM and not more than about 250 mM, 200 mM, 150 mM, 100 mM, and may be around 80 mM. Suitable buffers for a physiological pH are known in the art, e.g. bis-tris, etc.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

Gene Inactivation.

As described above, the coding sequence for rnb can be “knocked-out” or otherwise inactivated in the chromosome of the organism that is the source for microbial extracts, by deletion of all or a part of the coding sequence; frame-shift insertion; dominant negative mutations, etc. The genomes of a number of organisms, including E. coli, have been completely sequenced, thereby facilitating the genetic modifications. For example, a markerless knockout strategy method is described by Arigoni et al. (1998) Nat Biotechnol 16(9):851-6. Mutations can be combined in a single organism through known techniques of gene transfer.

A preferred method for inactivating targeted genes is described by Hoang et al. (1998) Gene 212:77-86. In this method, gene replacement vectors are employed that contain a tetracycline resistance gene and a gene encoding levan sucrase (sacB) as selection markers for recombination. The target gene is first cloned and mutagenized, preferably by deleting a significant portion of the gene. This gene is then inserted by ligation into a vector designed for facilitating chromosomal gene replacement. The E. coli cells are then transformed with those vectors. Cells that have incorporated the plasmid into the chromosome at the site of the target gene are selected, then the plasmid is forced to leave the chromosome by growing the cells on sucrose. Sucrose is toxic when the sacB gene resides in the chromosome. The properly mutated strain is selected based on its phenotype of tetracycline sensitivity and sucrose resistance. PCR analysis or DNA sequencing then confirms the desired genetic change. Alternatively, the method described by Datsenko and Wanner (2000) may be used, as shown in Example 1.

A gene of interest for deletion includes the ribonuclease II gene (rnb) of E. coli, which sequence may be found in Zilhao et al. (1993) Mol. Microbiol. 8 (1), 43-51; Genbank accession no. X67913. Using publicly available genetic sequences, the activity of the ribonuclease may be inactivated in a modified bacterial cell, as described above.

Alternatively the rnb protein may be removed from a cell extract by affinity methods. For example, an antibody or antibody fragment (e.g., Fab or scFv) is selected for specific affinity for the target enzyme using phage display or other well developed techniques. That antibody or antibody fragment is then immobilized on any of several purification beads or resins or membranes using any of several immobilization techniques. The immobilized antibody is contacted with the cell extract to bind to the target enzyme, and the immobilized antibody/enzyme complex then removed by filtration or gentle centrifugation.

In another example, the coding sequence of the targeted rnb protein may be modified to include a tag, such as the Flag® extension (developed by Immunex Corp. and sold by Stratagene), or a poly-histidine tail. Many other examples have been published and are known to those skilled in the art. The tagged proteins are then removed by passage over the appropriate affinity matrix or column. The amino acid extension and binding partner are chosen so that only specific binding occurs under conditions compatible with the stability of the cell extract, and without significantly altering the chemical composition of the cell extract.

In yet another example, the target enzyme or enzymes are separated by any of several methods commonly used for protein purification, such as substrate affinity chromatography, ion exchange chromatography, hydrophobic interaction chromatography, electrophoretic separation, or other methods practiced in the art of protein purification.

Expression Constructs.

The methods of the invention may make use of constitutive or regulated expression of various coding sequences for proteins that increase the synthetic capability of the cell-free reaction mix. The coding sequences may comprise, without limitation, one or more of AckA, EF-Tu, HchA, IbpA/B and IF 1-3 (see, for example, E. coli ackA gene: Genbank accession number M22956, Matsuyama et al. (1989) J. Bacteriol. 171 (1), 577-580; Hsp31 (HchA): Genbank accession number NP_{—}416476, Bury-Mone et al. (2009) PLoS Genet. 5 (9), E1000651; IbpA: Genbank accession number NP_{—}418142.1; IbpB: Genbank accession number NP_{—}418141.2; EfTu: Genbank accession number NP_{—}418407.1). As discussed above, the coding sequences may be expressed in the organism that is the source for microbial extracts. Alternatively the coding sequence is expressed in an exogenous system, either cellular or cell-free, and added to the reaction mixture as a crude extract or as a purified or semi-purified state.

Typically where supplemented expression is indicated, the concentration of the expressed protein in the cell or cell extract is at least 2-fold basal levels; at least about 10-fold basal levels: at least about 25-fold basal levels: at least about 50-fold basal levels; or more.

Expression and cloning vectors usually contain a promoter that is recognized by the host organism and is operably linked to the coding sequence of interest. Promoters are untranslated sequences located upstream (5′) to the start codon of a structural gene that control the transcription and translation of particular nucleic acid sequence to which they are operably linked. At this time a large number of promoters recognized by a variety of potential host cells are well known. While the native promoter may be used, for most purposes heterologous promoters are preferred, as they generally permit greater transcription and higher yields.

Promoters suitable for use with prokaryotic hosts include the p-lactamase and lactose promoter systems, alkaline phosphatase, a tryptophan (trp) promoter system, and numerous hybrid promoters such as the tac promoter. However, other known bacterial or bacteriophage promoters are also suitable, e.g. the lacI promoter, the lacZ promoter, the T3 promoter, the T7 promoter, the arabinose promoter, the gpt promoter, the lambda PR promoter, the lambda PL promoter, promoters from operons encoding glycolytic enzymes such as 3-phosphoglycerate kinase (PGK), and the acid phosphatase promoter. Their nucleotide sequences have been published, thereby enabling a skilled worker operably to ligate them to a sequence of interest using linkers or adaptors. Promoters for use in bacterial systems also will contain a Shine-Dalgamo (S.D.) sequence operably linked to the coding sequence.

Vectors useful for the transformation of an isolated DNA fragment encoding a protein of the invention into suitable host cells, or for cell-free expression are well known in the art. Typically the vector contains sequences directing transcription and translation of the relevant gene, a selectable marker, and sequences allowing autonomous replication or chromosomal integration. Vectors may also be used which promote the integration of the construct into the host cell genome. One or multiple copies may be integrated into a host cell genome

Cell Free Synthesis:

As used herein refers to the cell-free synthesis of macromolecules, usually protein translation, in a reaction mix comprising microbial cell extracts. The reaction mix will comprise an energy source; a template for production of the macromolecule, e.g. DNA, mRNA, etc.; amino acids, and such co-factors, enzymes and other reagents that are necessary for the synthesis, e.g. ribosomes, tRNA, polymerases, transcriptional factors, etc. As discussed above, the reaction mix may include pH buffers, may be supplemented with amino acids and/or nucleotides as required, and with respect to the present invention will comprise a microbial cell extract that is modified in the composition of proteins.

The reaction mixture will also include nucleotides to serve as energy carriers and as building blocks for nucleic acids. Although the triphosphate forms are required, the nucleotides can be added with any number of phosphate groups attached as long as the reaction mixture is activated to convert those forms into the triphosphate forms. These reagents are typically added from at least about 0.1 mM, at least about 0.25 mM, at least about 0.5 mM, and not more than about 2 mM concentrations.

In one example of a reaction mixture, glucose is added at about 20 to about 50 mM concentrations to be processed through central catabolism thereby regenerating the ATP, GTP, CTP, and UTP required for transcription and translation. In other examples, glucose or glycolytic or TCA cycle intermediates are slowly fed to a batch system, or the system operated as a continuous system using methods known in the art with, for example, from about 10 mM to about 50 mM glucose in the feed solution.

Such synthetic reaction systems are well-known in the art, and have been described in the literature. The cell-free synthesis reaction may be performed as batch, continuous flow, or semi-continuous flow, as known in the art.

Glucose or glycolytic intermediate energy source, as used herein, refers to compounds that provide energy for the synthesis of ATP from ADP, and which are part of the glycolytic pathway. These energy sources include glucose, glucose-1-phosphate, glucose-6-phosphate, fructose-6-phosphate, fructose-1,6-diphosphate, triose phosphate, 3-phosphoglycerate, 2-phosphoglycerate, phosphoenol pyruvate (PEP) and pyruvate. Preferred energy sources are PEP, pyruvate, and glucose-6-phosphate.

The energy source may be supplied as a suitable biologically acceptable salt or as the free acid, e.g. pyruvic acid, where applicable. The final concentration of energy source at initiation of synthesis will usually be at least about 5 mM, more usually at least about 10 mM, at least about 20 mM, and not more than about 1000 mM, usually not more than about 100 mM. Additional amounts may be added to the reaction mix during the course of synthesis to provide for longer reaction times.

In some embodiments the reaction mixture will comprise nucleotide triphosphates at a concentration of less than about 2.5 mM, and an energy source lacking high energy phosphate bonds, e.g. glucose or glycolytic intermediates at a concentration of at least about 10 mM.

Cell Extracts.

For the purposes of this invention, biological extracts are any preparation comprising the components of protein synthesis machinery, usually a bacterial cell extract, wherein such components are capable of translating a nucleic acid encoding a desired protein. Thus, a bacterial extract comprises components that are capable of translating messenger ribonucleic acid (mRNA) encoding a desired protein, and optionally comprises components that are capable of transcribing DNA encoding a desired protein. Such components include, for example, DNA-directed RNA polymerase (RNA polymerase), any transcription activators that are required for initiation of transcription of DNA encoding the desired protein, transfer ribonucleic acids (tRNAs), aminoacyl-tRNA synthetases, 70S ribosomes, N10-formyltetrahydrofolate, formylmethionine-tRNAfMet synthetase, peptidyl transferase, initiation factors such as IF-1, IF-2 and IF-3, elongation factors such as EF-Tu, EF-Ts, and EF-G, release factors such as RF-1, RF-2, and RF-3, and the like.

For convenience, the organism used as a source of extracts may be referred to as the source organism. Methods for producing active extracts are known in the art, for example they may be found in Pratt (1984), Coupled transcription-translation in prokaryotic cell-free systems, p. 179-209, in Hames, B. D. and Higgins, S. J. (ed.), Transcription and Translation: A Practical Approach, IRL Press, New York. Kudlicki at al. (1992) Anal Biochem 206(2):389 93 modify the S30 E. coli cell-free extract by collecting the ribosome fraction from the S30 by ultracentrifugation. An improved method is described by Liu et al. (2005) Biotech Progr 21:460-465.

In certain embodiments of the invention, the reaction mixture comprises extracts from bacterial cells, e.g. E. coli S30 extracts, as is known in the art. May different types of bacterial cells have been used for these purposes, e.g. Pseudomonas sp., Staphylococcus sp., Methanococcus sp., Methanobacterium sp., Methanosarcina sp., etc.

### Methods and Systems for Cell-Free Protein Synthesis

Cell-free synthesis, as used herein, refers to the cell-free synthesis of biological macromolecules in a reaction mix comprising biological extracts and/or defined reagents. The reaction mix will comprise a template for production of the macromolecule, e.g. DNA, mRNA, etc.; monomers for the macromolecule to be synthesized, e.g. amino acids, etc., and such co-factors, enzymes and other reagents that are necessary for the synthesis, e.g. ribosomes, tRNA, polymerases, transcriptional factors, etc., many of which are provided by the microbial cell extract. Such synthetic reaction systems are well-known in the art, and have been described in the literature. The system can be run under aerobic and anaerobic conditions.

In one embodiment of the invention, the reaction chemistry is as described in International Application WO 2004/016778, herein incorporated by reference. Oxidative phosphorylation is activated, providing for increased yields and enhanced utilization of energy sources. Improved yield is obtained by a combination of factors, including the use of biological extracts derived from bacteria grown on a glucose containing medium; an absence of polyethylene glycol; and optimized magnesium concentration. This provides for a homeostatic system, in which synthesis can occur even in the absence of secondary energy sources.

The compositions and methods of this invention allow for production of proteins with any secondary energy source used to energize synthesis. These can include but are not limited to glycolytic intermediates, such as glucose, pyruvate, or acetate. Other glycolytic intermediates, such as glucose-6-phosphate, fructose-6-phosphate, fructose-1,6-diphosphate, triose phosphate, 3-phosphoglycerate, 2-phosphoglycerate, and phosphoenolpyruvate (PEP), are already phosphorylated, so they may not be susceptible to phosphate limitation. Any compound used to generate reduction equivalents or to activate a pathway that may generate reduction equivalents may also be added. These compounds include amino acids (particularly glutamate), tricarboxylic acid (TCA) cycle intermediates (citrate, cis-aconitate, isocitrate, α-ketoglutarate, succinyl-CoA, succinate, fumarate, malate, and oxaloacetate), or other molecules that can be directed into central metabolism (such as glyoxylate). In addition, vesicles containing respiratory chain components may also be added to assist in energy generation. The energy source may be supplied in concentrations around 30 mM. The secondary energy sources are not usually added in concentrations greater than 150 mM. Additional amounts of the energy source may be added to the reaction mixture during the course of protein expression to fuel longer reaction times.

The template for cell-free protein synthesis can be either mRNA or DNA. Translation of stabilized mRNA or combined transcription and translation converts stored information into protein. The combined system, generally utilized in E. coli systems, continuously generates mRNA from a DNA template with a recognizable promoter. Either endogenous RNA polymerase is used, or an exogenous phage RNA polymerase, typically T7 or SP6, is added directly to the reaction mixture. Alternatively, mRNA can be continually amplified by inserting the message into a template for QB replicase, an RNA dependent RNA polymerase. Purified mRNA is generally stabilized by chemical modification before it is added to the reaction mixture. Nucleases can be removed from extracts to help stabilize mRNA levels. The template can encode for any particular gene of interest.

Other salts, particularly those that are biologically relevant, such as manganese, may also be added. Potassium is generally added between 50-250 mM and ammonium between 0-100 mM. The pH of the reaction is generally run between pH 6-9. The temperature of the reaction is generally between 20° C. and 40° C. These ranges may be extended.

Vesicles, either purified from the host organism or synthetic, may also be added to the system. These may be used to enhance protein synthesis and folding. This cytomim technology has been shown to activate processes that utilize membrane vesicles containing respiratory chain components for the activation of oxidative phosphorylation. The present methods may be used for cell-free expression to activate other sets of membrane proteins.

The reactions may be large scale, small scale, or may be multiplexed to perform a plurality of simultaneous syntheses. Additional reagents may be introduced to prolong the period of time for active synthesis. Synthesized product is usually accumulated in the reactor, and then is isolated and purified according to the usual methods for protein purification after completion of the system operation.

Of particular interest is the translation of mRNA to produce proteins, which translation may be coupled to in vitro synthesis of mRNA from a DNA template. Such a cell-free system will contain all factors required for the translation of mRNA, for example ribosomes, amino acids, tRNAs, aminoacyl synthetases, elongation factors and initiation factors. Cell-free systems known in the art include E. coli extracts, etc., which can be treated with a suitable nuclease to eliminate active endogenous mRNA.

In addition to the above components such as cell-free extract, genetic template, and amino acids, materials specifically required for protein synthesis may be added to the reaction. These materials include salts, polymeric compounds, cyclic AMP, inhibitors for protein or nucleic acid degrading enzymes, inhibitors or regulators of protein synthesis, oxidation/reduction adjusters, non-denaturing surfactants, buffer components, spermine, spermidine, putrescine, etc.

The salts preferably include potassium, magnesium, ammonium and manganese salts of acetic acid or sulfuric acid, and some of these may have amino acids as a counter anion. The polymeric compounds may be polyethylene glycol, dextran, diethyl aminoethyl dextran, quaternary aminoethyl and aminoethyl dextran, etc. The oxidation/reduction adjuster may be dithiothreitol, ascorbic acid, glutathione and/or their oxides. Also, a non-denaturing surfactant such as Triton X-100 may be used at a concentration of 0-0.5 M. Spermine, spermidine, or putrescine may be used for improving protein synthetic ability, and cAMP may be used as a gene expression regulator.

When changing the concentration of a particular component of the reaction medium, that of another component may be changed accordingly. For example, the concentrations of several components such as nucleotides and energy source compounds may be simultaneously controlled in accordance with the change in those of other components. Also, the concentration levels of components in the reactor may be varied over time.

Preferably, the reaction is maintained in the range of pH 5-10 and a temperature of 20°-50° C., and more preferably, in the range of pH 6-9 and a temperature of 25-40° C.

The amount of protein produced in a translation reaction can be measured in various fashions. One method relies on the availability of an assay that measures the activity of the particular protein being translated. Examples of assays for measuring protein activity are a luciferase assay system, and a chloramphenical acetyl transferase assay system. These assays measure the amount of functionally active protein produced from the translation reaction. Activity assays will not measure full-length protein that is inactive due to improper protein folding or lack of other post translational modifications necessary for protein activity.

Another method of measuring the amount of protein produced in a combined in vitro transcription and translation reactions is to perform the reactions using a known quantity of radiolabeled amino acid such as ³⁵S-methionine or ¹⁴C-leucine and subsequently measuring the amount of radiolabeled amino acid incorporated into the newly translated protein. Incorporation assays will measure the amount of radiolabeled amino acids in all proteins produced in an in vitro translation reaction including truncated protein products. The radiolabeled protein may be further separated on a protein gel, and by autoradiography confirmed that the product is the proper size and that secondary protein products have not been produced.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the cell lines, constructs, and methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

Using the platform described by Woodrow, K. A., Swartz, J. R., A sequential expression system for high-throughput functional genomic analysis. Proteomics 2007, 7, 3870-3879, gene products that affect protein accumulation and activation in cell-free translation systems were evaluated. Briefly, linear DNA expression templates (ETs) for each open reading frame of the chosen organism are constructed using a single two-staged PCR reaction. The ETs are used to direct the cell-free transcription and translation of their cognate protein in a single well of a 96-well microtiter plate. This creates an array of cell extracts individually enriched with a single gene product, which is then tested for expression of a reporter protein.

This platform was employed for the functional genomic analysis of Escherichia coli (E. coli) strain K-12. Approximately 80 proteins were identified that significantly enhanced protein expression, and approximately 60 proteins that inhibited protein expression, including the following:

TABLE 1
Examples of proteins that could influence protein accumulation
by impacting on a variety of metabolic activities.

| | |
|---|---|
| Metabolic Function or Target | Observed Effect |

Energy supply/regeneration

| | |
|---|---|
| Energy pathway enzymes | |
| [symbol] GdhA glutamate dehydrogenase | Postive |
| [symbol] Mdh malate dehydrogenase | Postive |
| Kinases | |
| [symbol] AckA propionate kinase/acetate kinase | Postive |
| [symbol] Ndk nucleoside diphosphate kinase | Postive |

Nucleic acid supply/stability

| | |
|---|---|
| Transcription factors | |
| [symbol] GreA transcription elongation factor | Postive |
| [symbol] CspF cold shock protein | Postive |
| [symbol] NusG transcription termination factor | Postive |
| Chaperones/inhibitors | |
| [symbol] CspC stress protein | Postive |
| [symbol] RraA ribonuclease E inhibitor protein A | Postive |
| [symbol] YrhC putative chaperone | Postive |
| Nucleases | |
| [symbol] Pnp polynucleotide phosphorylase | Negative |
| [symbol] Rna ribonuclease I | Negative |
| [symbol] MazF toxin with ribonuclease activity | Negative |

Amino acid supply

| | |
|---|---|
| Amino acid degradation/modification | |
| [symbol] YqeA predicted amino acid kinase | Negative |
| [symbol] TdcB catabolic threonine dehydratase | Negative |
| [symbol] CadA lysine decarboxylase | Negative |
| [symbol] LysC aspartate kinase | Negative |

Protein expression

| | |
|---|---|
| Initation factors | |
| [symbol] InfA protein chain initiation factor IF-1 | Postive |
| [symbol] InfB protein chain initiation factor IF-2 | Postive |
| [symbol] InfC protein chain initiation factor IF-3 | Postive |
| [symbol] YciH translation initiation factor | Postive |
| Elongation factors | |
| [symbol] TufA & TufB elongation factor Tu | Postive |
| [symbol] Tsf protein chain elongation factor Ts | Postive |
| [symbol] FusA elongation factor G | Postive |
| [symbol] LepA elongation factor 4 | Postive |

Protein stability

| | |
|---|---|
| Proteases | |
| [symbol] PtrA protease III | Negative |
| [symbol] DegP serine protease Do | Negative |
| [symbol] ClpB chaperone | Negative |
| [symbol] YpdF aminopeptidase | Negative |

Protein folding

| | |
|---|---|
| Chaperones | |
| [symbol] HchA Hsp31 molecular chaperone | Postive |
| [symbol] YciM putative heat shock protein | Postive |
| [symbol] CspG cold shock protein | Postive |
| [symbol] DjlC Hsc56, co-chaperone of Hsc62 | Postive |

A statistical design of experiment (DoE) approach was adopted to identify combinations of positive effectors that exhibit cooperative interactions, thus further enhancing protein production. While improving the productivity and protein yields of the CFPS system, it was discovered that the addition of targeted biocatalysts was not sufficient to achieve maximum protein production. Specifically, re-optimization of small molecule metabolite concentrations was utilized to create a CFPS system suitable for enhanced productivity.

To overcome substrate limitation, a semi-fed-batch approach was adopted, in which the reaction mixtures were replenished with the depleting small molecules every 2 hr of the incubation period. With this mode, the period of protein production was extended, and GFP accumulation was enhanced by ˜25% over the observed plateau concentration.

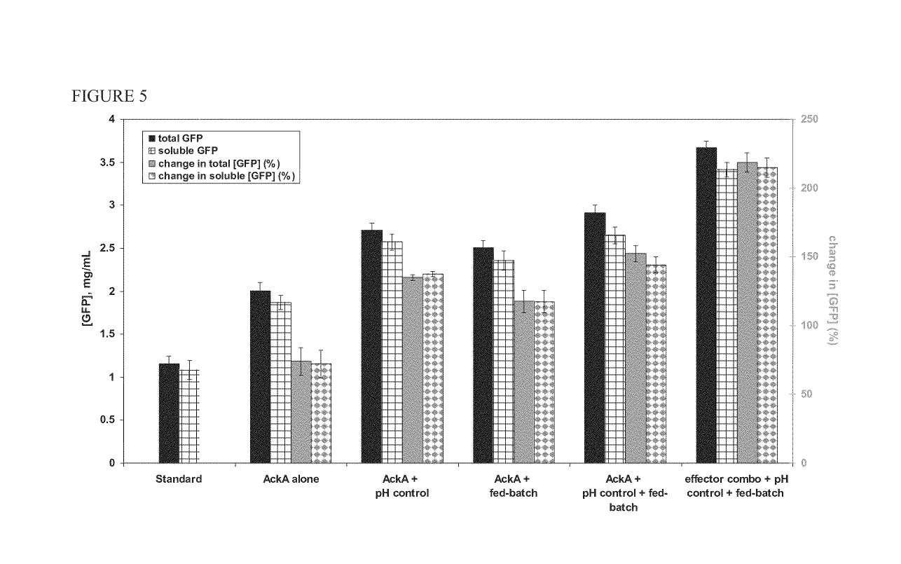

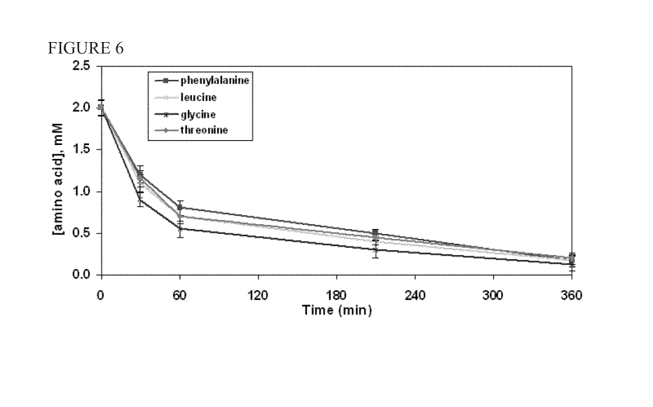

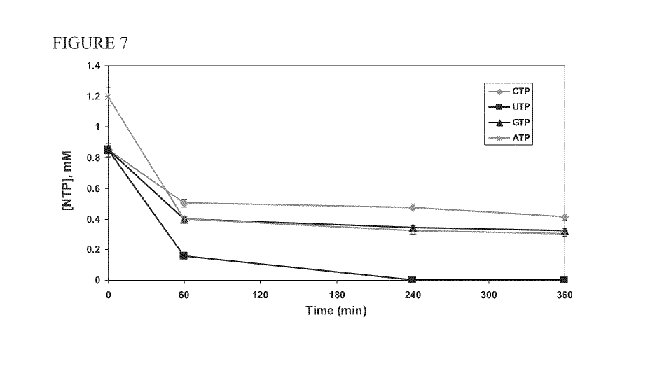

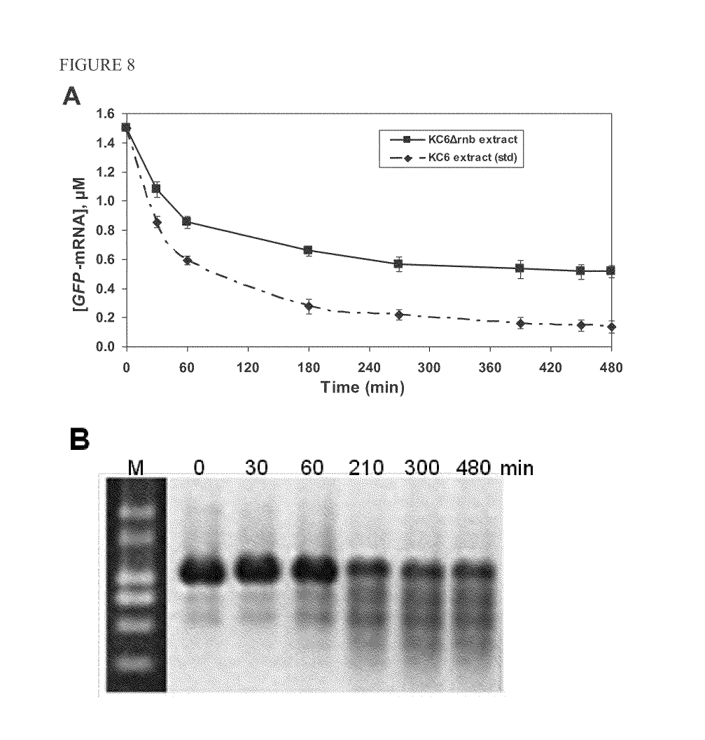

This modified system (with pH control and replenishment of depleting small molecules) was then used to perform the DoE. The statistical analysis identified AckA as the most significant single effector, improving GFP yields by ˜150% over the standard system, and a set of 8 proteins as the most influential combination, which enhanced protein production by ˜220% over the standard system (FIG. 5). The 8-effector combination consisted of gene products that influenced diverse metabolic areas, including energy regeneration (AckA), protein folding (heat-shock protein HchA small heat-shock proteins IbpA and IbpB), translation initiation (initiation factors IF1, IF2, and IF3), and translation elongation (elongation factor EF-Tu).

TABLE 2
Positive effectors that were purified for statistical design of experiment (DoE). (n = 3)

| | Size and | | Change in [protein] |
|---|---|---|---|
| Protein | Subunit Composition | Metabolic Role | from Baseline (%) |
| Acetate kinase AckA | 86 kDa, Dimer | Energy Supply | 72 ± 12 |
| Cytidine deaminase Cdd | 64 kDa, Dimer | Transcription, | 50 ± 8 |
| | | Energy Supply | |
| Glutamate dehydrogenase GdhA | 300 kDa, Hexamer | Energy Supply | 24 ± 3 |
| GroEL/GroES chaperones | 870 kDa, 21-mer | Protein Folding | 49 ± 8 |
| Elongation factor Tu EF-Tu | 43 kDa, Monomer | Translation | 37 ± 3 |
| Pyruvate decarboxylase AceE | 200 kDa, Dimer | Energy Supply | 28 ± 3 |
| Elongation factor 4 LepA | 74 kDa, Monomer | Translation | 66 ± 7 |
| Nucleoside diphosphate kinase Ndk | 64 kDa, Tetramer | Energy Supply | 57 ± 8 |
| Heat-shock protein 31 HchA | 62 kDa, Dimer | Protein Folding | 43 ± 6 |
| Small heat-shock protein IbpA | 16 kDa, Monomer | Protein Folding | 32 ± 4 |
| Small heat-shock protein IbpB | 650 kDa, 40-mer | Protein Folding | 64 ± 4 |
| Initiation factor IF-1 | 8 kDa, Monomer | Translation | 12 ± 1 |
| Initiation factor IF-2 | 97 kDa, Monomer | Translation | 15 ± 3 |
| Initiation factor IF-3 | 21 kDa, Monomer | Translation | 8 ± 1 |

The cell-free expression system was further modified by targeting the genes of highly-negative effectors [polynucleotide phosphorylase (pnp), ribonuclease II (rnb), ribosome-associated inhibitor (raiA), and nucleoside triphosphate pyrophosphohydrolase (mazG)] for deletion from the standard CFPS extract source cell genome. Single gene-deletion strains were constructed to insert markerless deletions into the chromosome and were used to prepare cell extracts. The rnb deletion strain was the sole mutation that influenced the in vitro expression system, causing a ˜70% increase in total and soluble GFP yields.

With these modifications of the conventional in vitro expression system [protein supplementation (AckA, EF-Tu, HchA, IbpA/B, IF 1-3), pH control, replenishment of limiting reaction substrates during incubation period, and stabilization of mRNA], the total and soluble yields of several proteins were increased by ˜300-400% (Table 3). The methods described by this invention provide for the coordinated improvement of the catalytic system and the small molecule metabolite environment to synergistically improve cell-free protein expression. The in vitro functional genomics platform, with which 1000s of proteins can be analyzed in parallel, allows for the rapid identification of effectors of the cell-free protein synthesis system as potential targets that could increase the productivity of CFPS. The improvement of cell free protein production then requires the methods described in this invention that combine combinatorial analysis of multiple effectors coupled with simultaneous enhancement of multiple molecular substrates needed for protein synthesis as well as modifications to stabilize the chemical status of the reaction system.

TABLE 3
Improved protein yields of several proteins in the modified CFPS system.

| | Standard System | | Modified System with KC6_rnb | |
|---|---|---|---|---|
| PROTEIN | Total (μg/mL) | Soluble (μg/mL) | Total (μg/mL) | Soluble (μg/mL) |
| GFP | 1153.7 ± 80.42 | 1084.2 ± 73.2 | 5018.1 ± 87.6 | 4717.0 ± 77.2 |
| CAT | 1201.6 ± 91.1 | 1129.7 ± 73.3 | 4489.7 ± 111.4 | 4237.8 ± 123.9 |
| _-Lactamase | 263.6 ± 32.4 | 232.4 ± 28.2 | 1239.2 ± 82.6 | 1046.8 ± 62.3 |
| Urokinase | 287.5 ± 19.6 | 181.5 ± 27.1 | 1062.9 ± 87.3 | 653.7 ± 57.8 |
| vtPA | 322.3 ± 21.7 | 213.4 ± 32.7 | 1194.4 ± 67.2 | 768.4 ± 32.5 |

The total and soluble yields of several proteins were enhanced ˜300-400% in the modified CFPS system [protein supplementation (AckA, EF-Tu, HchA, IbpA/B, IF 1-3); pH control; and replenishment of limiting reaction substrates during incubation period] using the S30 cell extract from the KC6Δrnb strain. (n=3)

### Example 2

### Functional Genomic Analysis of Escherichia coli Using a Sequential Cell-Free Protein Synthesis Platform

To overcome the limitations of current proteomic methods, protein synthesis and folding was used as a single indicator in the context of complete cell extracts, which allows for a single survey that is capable of providing functional information for hundreds of gene products. Based on this principle, we developed a sequential CFPS platform that is capable of characterizing a variety of diverse proteins in the context of the dynamic metabolic networks that exist in vivo. In a 96-well format, the first round of CFPS creates an array of cell extracts each individually enriched with a single gene product. These first-round products are expressed in-parallel from linear DNA expression templates under reaction conditions that are conducive for general protein activation.

Following the first-round expression, the linear templates are selectively degraded, and a second template for a reporter enzyme is added in order to initiate a subsequent round of protein expression. Reporter concentration and activity identifies first-round gene products directly affecting protein expression, stability, and activation as well as amino acid and nucleic acid stability and the CFPS energy supply.

With this method, combined transcription, translation, and protein folding, which is arguably the most central and most complex metabolic system, serves as a single assay. The developed experimental system also includes the central catabolic pathways of glycolysis, the TCA cycle, and oxidative phosphorylation in order to fuel the energy intensive reactions of protein synthesis in a reaction of several hours duration. In this way, a major portion of central metabolism is also probed. There are several other key features of this CFPS platform that make it an attractive tool for genome-wide functional analysis. These include:

(1) The dilute catalytic environment of the cell extract, which is approximately 10- to 20-fold less concentrated than the cytoplasm. The significantly reduced rates of protein synthesis and folding allow the effects of overexpressed translation (initiation and elongation) factors and chaperones to be better detected.

(2) With the inactivation of the native RNA polymerase in the cell extract and the nearly constant composition of the enzymatic mixture, the risk of misleading secondary effects is much lower. Any detected changes can be attributed to direct effects of the gene product being tested.

(3) By simulating the cellular situation in which external energy sources are not available, our in vitro protein expression system essentially replicates metabolism in a cell that must alter its metabolism by using internal energy stores to either produce enzymes needed to utilize a new external energy source or to prepare for stationary phase.

(4) The activation of oxidative phosphorylation allows us to study the metabolic transfer of electrons from primary electron donors to oxygen. However, since the focus of the proposed system is on gene products that directly influence intracellular metabolism, factors regulating gene expression will not be detected. While this is a limitation, it also confers the advantage that confusing results produced by gene induction and the activities of secondary gene products will be avoided.

We conducted a genome-wide survey of E. coli for non-membrane gene products that have an effect on the cell-free metabolism. The sequential rounds of protein expression allowed us to identify 139 effectors (79 positive and 60 negative) that influence the in vitro transcription, translation, and protein folding of our reporter proteins, as well as energy metabolism and RNA and protein stability in the system. Encouragingly, most of the observed effects are consistent with the reported in vivo metabolic functions of the gene products. This genome-wide survey of E. coli can be characterized as an unbiased or “discovery-oriented” approach to functional genomics, for the analyte-enriched CFPS systems were analyzed with the purpose of identifying as many effectors as possible without imposing previous knowledge on the experimental design. By employing this discovery-oriented strategy, many predicted proteins, as well as several unanticipated effectors, were observed to affect protein expression and activity.

### Materials and Methods

Media components and chemical reagents were the highest available purity and were purchased from Sigma-Aldrich (St. Louis, Mo.) unless indicated otherwise. Platinum^{R} PCR SuperMix and Accuprime Pfx DNA polymerase (DNAP) were purchased from Invitrogen (Carlsbad, Calif.). Oligonucleotides were synthesized by Sigma-Aldrich (St. Louis, Mo.) and the Protein and Nucleic Acid Facilities (Stanford University, Stanford, Calif.). dNTPs and restriction enzymes were purchased from New England Biolabs (NEB, Ipswich, Mass.). Isolation and purification of DNA was performed using the QIAquick™ 96 PCR purification, QIAquick™ gel extraction, and plasmid maxiprep kits (Qiagen Ltd., Valencia, Calif.). T7 RNA polymerase (RNAP) and DsbC were prepared by overexpression and purification from E. coli strains BL21 (pAR1219) and BL21(DE3) (pETDsbChisC), respectively, as described previously.

Generation of Transcriptionally-Active Linear DNA Templates Using PCR.

The linear DNA expression templates (ETs) that were used to direct the first round expression of the effector candidates in the sequential CFPS reactions were generated as described elsewhere with minor modifications. Genomic DNA was prepared from E. coli strain A19 using DNeasyR Blood and Tissue kit (Qiagen Ltd., Valencia, Calif.). The open-reading frames (ORFs) for the gene products were amplified from the purified genomic DNA using gene-specific primers designed with OligoPerfect Designer (Invitrogen, Carlsbad, Calif.). Each ORF was amplified in a 50-μL PCR reaction containing 45 μL of PlatinumR PCR SuperMix (1 U Taq DNAP, 20 mM Tris-HCl (pH 8.4), 50 mM KCl, 1.5 mM MgCl₂, and 200 μM dNTPs), 1 μM each of the sense and antisense primers, and 2 μg/mL gDNA. The gene targets were amplified beginning with a single incubation at 94° C. for 2 min, followed by 25 cycles of 94° C. for 15 s, 60° C. for 30 s, and 72° C. for 5 min. Difficult targets were amplified by lowering the annealing temperature to 55° C. The PCR products were purified using the QIAquick™ 96 PCR purification kit according to the manufacturer's instructions. Yields for each ORF were quantified densitometrically against a low DNA mass ladder (Invitrogen, Carlsbad, Calif.) using ethidium bromide stained agarose E-GelR 96 gels (1%, w/v) (Invitrogen, Carlsbad, Calif.), followed by analysis using Bio-Rad Quantity OneR software (Hercules, Calif.), in some cases. The gel images were manipulated by aligning and arranging the lanes using the Invitrogen EEditor TM software (Carlsbad, Calif.). Each gene-specific sense primer was extended at the 5′-terminus with the sequence 5′-GTTTAACTT AAGAAGGAGA TATACAT-3′, whereas each gene-specific antisense primer was extended at the 5′-terminus with the sequence 5′-CAGCGGTGGC AGCAGCCAAC TCA-3′. The nucleotide extensions introduced complementarity between the gene coding sequence and the transcription regulatory elements that were added in the next step.

The bacteriophage T7 promoter (PT7.sp3) and terminator (Term.sp3) elements were amplified from the pK7CAT plasmid. The 250-basepair (bp) PT7.sp3 was amplified using the GC-rich FwdPT7 sense primer, 5′-ATGCAGGTCA TCCGAGGGGT TAACGAGTTC GCGGCCGCTT AGGCACCCCA GGCTTTAC-3′, and the RevPT7 antisense primer, 5′-CATATGTATA TCTCCTTCTT AAAGTTAAAC AAAATGATCT CTAGATCG AAACCGTTGT GGTCTC-3′. The 170-bp Term.sp3 was amplified using the FwdTERM sense primer, 5′-TGAGTTGGCT GCTGCCACCG CTG-3′, and the GC-rich RevTERM antisense primer: 5′-ATGCA GGTCATCCGA GGGGTTAACG AGTTCGACGA GCGTCAGCTT GCATGCCCTG CAGCT-3′. Underlined regions denote sequence complementarity to extensions flanking each gene coding sequence. The regulatory elements were each amplified in a total volume of 50 μL by combining 45 μL of PlatinumR PCR SuperMix (1 U Taq DNAP, 20 mM Tris-HCl (pH 8.4), 50 mM KCl, 1.5 mM MgCl₂, and 200 μM dNTPs), 1 μM each of the appropriate sense and antisense primers, and 2 μg/mL pK7CAT.PT7.sp3 and Term.sp3 were amplified using the following temperature cycles: one cycle of 94° C. for 2 min, followed by 25 cycles of 94° C. for 30 s, 60° C. for 30 s, and 72° C. for 30 s. The PCR products were separated in ethidium bromide-stained 2% (w/v) agarose E-Gel® gels, and the products were recovered using the QIAquick™ gel extraction kit (Qiagen Ltd., Valencia, Calif.). The double-stranded DNA (dsDNA) products were eluted from the capture column using 50 mL of water, and the concentration and purity of both regulatory elements were determined by gel analysis and by measuring the absorbance at 260 and 280 nm.

The extension and GC-rich single primer amplification reactions were performed in a total volume of 50 μL by combining 5-10 nM purified ORF template from the first PCR, 30 nM each of PT7.sp3 and Term.sp3 (˜3 μL each of the PT7.sp3 and Term.sp3 preparations described above), and 45 μL of PlatinumR PCR SuperMix (1 U Taq DNAP, 20 mM Tris-HCl (pH 8.4), 50 mM KCl, 1.5 mM MgCl₂, and 200 μM dNTPs). The thermal cycler was programmed to perform the following sequence: one incubation at 94° C. for 2 min; 10 cycles of 94° C. for 30 s, 57° C. for 1 min, and 72° C. for 5 min; and 15 cycles of 94° C. for 30 s, 67° C. for 1 min, and 72° C. for 5 min. After the first 10 cycles at the lower annealing temperature, 20 μM SP3 GC-rich primer (5′-ATGCAGGTCA TCCGAGGGGT T-3′) was added to each PCR reaction tube and mixed by inverting or gentle vortexing, and the remaining 15 temperature cycles were completed at the higher annealing temperature (Ta=67° C.). The full-length ETs were purified using the QIAquick™ 96 PCR purification kit according to the manufacturer's instructions. Yields for each template were quantified densitometrically against a low DNA mass ladder using ethidium bromide-stained agarose E-GelR 96 gels (1%, w/v), followed by analysis using Bio-Rad Quantity OneR software, in some cases. The gel images were manipulated by aligning and arranging the lanes using the Invitrogen E-Editor™ software (Carlsbad, Calif.).

Batch Cell-Free Protein Synthesis (CFPS) Reactions in Microtiter Plates Using Linear or Plasmid Expression Templates (ETs)

Batch CFPS reactions were conducted using a modified Cytomim system with glutamate and oxidative phosphorylation as the energy source. A standard 15-μL reaction mixture contained 1.2 mM ATP, 0.86 mM each of GTP, CTP, and UTP, 130 mM potassium glutamate, 10 mM ammonium glutamate. 8 mM magnesium glutamate, 10 mM potassium phosphate (pH 7.2), 34 μg/mL folic acid, 171 μg/mL E. coli tRNA (Roche Applied Sciences, Indianapolis, Ind.), 100 μg/mL T7 RNAP, 2 mM each of 20 unlabeled amino acids, 0.17 mM nicotinamide adenine dinucleotide (NAD+), 0.26 mM coenzyme A (CoA), 4 mM sodium oxalate, 1.5 mM spermidine, 1 mM putresceine, 4 mM GSSG, 1 mM GSH, 75 μg/mL DsbC, ˜13.3 μg/mL linear or plasmid template, and 0.24 volumes of KGK10S30 extract, which was incubated with 50 μM iodoacetamide (IAM) for 30 min at room temperature before being used in the reaction system. L-[¹⁴C]-Leucine (Amersham GE Biosciences, Piscataway, N.J., USA) (4.2 μM) was not added, unless stated otherwise.

Preparation of the KGK10 S30 cell extract has been described elsewhere. All standard CFPS reactions were supplemented with vitamins, cofactors, and trace metal ions in order to maximize activation of the translated proteins. The concentrations of these components had been optimized for maximal production and activation of a library of cofactor-dependent enzymes. Thiamin, riboflavin, coenzyme B12, pyridoxal 5′-phosphate, biotin, and lipoic acid were added at 20 μM; flavin adenine dinucleotide (FAD) at 50 μM. The trace metal cocktail introduced ferric(III) chloride, cobalt(II) chloride, and boric acid to a final concentration of 250 μM each; cupric(III) sulfate at 60 μM; and manganese(II) sulfate and zinc(II) sulfate at 30 μM. Sodium molybdate(VI) was added separately at 250 μM. Standard 15-μL batch CFPS reactions were carried out in covered tissue culture treated, flat-bottom 96-well microtiter plates made of polystyrene (BD Falcon™ #353936. Franklin Lakes, N.J.) and incubated in a humidified incubator at 37° C. for 5 hrs.

If L-[¹⁴C]-leucine was added to the reaction mixture, total protein accumulation was estimated by determining the incorporation of the labeled amino acid into TCA precipitable counts: 5 μL of the CFPS reaction mixture was spotted onto a piece of filter paper immediately after the incubation period, and the TCA-insoluble radioactivity was measured using a liquid scintillation counter (Wallac 1450 Microbeta LSC, Perkin Elmer), as described elsewhere. In order to measure the soluble fraction of the product protein, the CFPS reaction was centrifuged at 12,000×g for 10 min and 4° C., and 5 μL of the supernatant was used to determine TCA-precipitable counts as described above.

Termination of First-Round Cell-Free Expression

The first round of expression directed by the linear ETs was terminated as described previously. The PT7.sp3 element was designed to contain two restriction sites (GATC) between the promoter and start-ATG that are recognized and cleaved by the enzyme DpnII. The unmethylated linear DNA templates were, therefore, vulnerable to DpnII digestion at these two sites, as well as other sites present in the gene coding sequence. This four-base site can be protected by methylation of the adenosine base by using an exogenous methylase or by using plasmid templates that have been prepared from a dam+n strain. DpnII was purchased from New England Biolabs (Ipswich, Mass., USA), and a 5-mL PD-10 (Sephadexm) desalting column (GE Healthcare Bio-Sciences, Piscataway, N.J.) was used to exchange the enzyme storage buffer (10 mM Tris-HCl (pH 7.4), 200 mM NaCl, 1 mM dithiothreitol (DTT), 0.1 mM ethylenediaminetetraacetic acid (EDTA), 200 μg/ml bovine serum albumin (BSA), and 50% glycerol) with the S30 buffer used in the CFPS reaction mixture. The enzyme was diluted 25-fold with 2.5 mL of S30 buffer (10 mM Tris-acetate (pH 8.2), 14 mM magnesium acetate, 60 mM potassium acetate, and 1 mM DTT) and was loaded onto the PD-10 column, which had been equilibrated with 25 mL of S30 buffer. DpnII was eluted from the column by addition of 3.5 mL of S30 buffer. The eluate was collected and concentrated to I U/μL with an AmiconR Ultra-15 centrifugal filter device having a 5-kDa molecular weight cut-off (MWCO) (Millipore, Billerica, Mass.) according to the manufacturer's instructions. The enzyme was stored in the presence of 0.5 mg/mL BSA up to two weeks at 4° C. The ability of DpnII to selectively terminate protein expression from the linear ETs had been investigated as previously reported by evaluating the digestion patterns of unmethylated PCR templates and methylated plasmid templates.

Sequential Cell-Free Protein Expression.

Sequential rounds of CFPS were conducted as described elsewhere. Briefly, the first-round expression was carried out in 15-μL batch reactions as described above with linear PCR templates and without the addition of L-[¹⁴C]-leucine. After 40 min of incubation at 37° C., the reaction plate was removed from the humidified incubator and placed on ice for 15 s to prevent evaporation. The first round of cell-free protein synthesis was terminated by digesting the unmethylated linear ET with 1 U DpnII at 37° C. for 15 min, after which the plate was again placed on ice for 15 s. The methylated plasmid template for the reporter protein (˜13.3 μg/mL) was added in a total volume of 1 μL to each reaction well, and the plate was returned to the 37° C. incubator for 4 hr. The methylated templates for the reporters were pK7CAT for chloramphenicol acetyltransferase, pK7-59236 for β-lactamase, and pK7vtPA for a variant of human tissue-type plasminogen activator. The total reporter activity in the reaction mixture was determined according to published methods as described below. L-[¹⁴C]-Leucine (4.2 μM) was not added at the beginning of the second round, unless noted otherwise. If introduced into the reaction mixture, the amount of total reporter protein produced was estimated by measuring TCA-insoluble radioactivity using a liquid scintillation counter (Wallac 1450 Microbeta LSC, Perkin Elmer) as described above.

Expression and Purification of IbpB E. Coli.

Small heat-shock protein (sHsp) IbpB was purified in its native (untagged) form. The protein sample was concentrated to 2 mg/mL using 300-kDa MWCO centrifugal devices (AmiconR Ultra-15, Millipore, Billerica, Mass.) according to the manufacturer's instructions. Protein concentration was determined using the DC Protein Assay (Bio-Rad, Hercules, Calif.) according to the manufacturer's instructions with bovine serum albumin as a standard. Protein activity was qualitatively assessed by supplementing the standard CFPS reaction with the purified sHsp and comparing the effects against those previously observed in sequential CFPS experiments. The purified proteins were stored in 10 mM potassium phosphate (pH 7.2) with 20% (v/v) sucrose. The protein solutions were flash-frozen in liquid nitrogen and stored at −20° C. until use. The sucrose from the purified IbpB sample was removed immediately before use by using centrifugal devices with 300-kDa molecular-weight cut-off (MWCO) membranes (Pall Corporation, New York, USA) according to the manufacturer's instructions. The sucrose was removed to prevent any potential CFPS inhibition, for such inhibition was previously observed in expression reactions containing ˜6% (v/v) sucrose.

Construction of Expression Plasmids.

Plasmid templates were constructed to direct the second-round CFPS of various E. coli enzymes (uridylate kinase, thioredoxin reductase, cytidine deaminase, malate dehydrogenase, and glucose 6-phosphate dehydrogenase). Standard procedures were used to subclone each gene into the expression vector pY71L, placing the coding region between the T7 promoter and T7 terminator. Each gene was amplified by polymerase chain reaction (PCR) using the purified E. coli genomic DNA as the template and a pair of gene-specific oligonucleotide primers. Each sense primer included the NdeI restriction site for subsequent fragment digestion and ligation with vector, whereas each antisense primer included the SphI site. Each gene was amplified in a 50-μL PCR reaction containing 2.5 U Accuprime Pfx DNAP, 1 μM each of the sense and antisense primers, 2 μg/mL genomic DNA, 300 μM dNTPs and MgSO4 to a final concentration of 1.3 mM. The targets were amplified in parallel beginning with a single incubation at 95° C. for 2 min, followed by 25 cycles of 95° C. for 15 sec, 60° C. for 30 sec. and 68° C. for 5 min. The PCR products were purified using QIAquick™ PCR purification kit (Qiagen Ltd., Valencia, Calif.) according to the manufacturer's instructions. The purified products and expression vector were subjected to NdeI/SphI digestion and subsequent separation in 1.2% (w/v) agarose gels. A gel extraction kit (Qiagen Ltd., Valencia, Calif.) was used to recover the desired digested products. The double-stranded DNA (dsDNA) products were eluted from the capture column using 50 μL of water, and the concentration and purity of the digested products were determined by gel analysis and by measuring the absorbance at 260 and 280 nm. Each purified digested gene product was ligated with the digested pY71L vector. The expression constructs isolated from cultures of individual transformants were screened by NdeI/SphI digestion and further verified by DNA sequencing (Pan and Nucleic Acid Facilities, Stanford University, Stanford, Calif.). The plasmid maxiprep kit (Qiagen Ltd., Valencia, Calif.) was used to purify the DNA templates for cell-free protein expression.

Enzymatic Activity Assays.

All spectrophotometric measurements were conducted using a SpectraMax® 190 microplate spectrophotometer (Molecular Devices, Sunnyvale, Calif.). Enzyme-specific activities were measured without purification and corrected for any detectable background activity by comparing to an equivalent volume of a CFPS reaction conducted without a DNA template.

Chloramphenicol Acetyltransferase.

The formation of reduced coenzyme-A (CoA) was monitored at 412 nm and 37° C. using the thiol reagent 5,5′-dithiobis (2-nitrobenzoic acid) (DTNB, Σ412=13.6 mM⁻¹ cm⁻¹) in order to determine CAT specific activity. The assay was conducted in a total volume of 0.2 mL containing 0.1 mM acetyl-CoA, 0.1 mM chloramphenicol, and 0.4 mg/mL DTNB in 100 mM Tris124 HCl (pH 7.8). The reaction was initiated by the addition of unpurified cell-free-expressed CAT to a final concentration of about 0.3 μg/mL (2 μL of ˜40-fold diluted CFPS reaction sample). Specific activity is expressed in units per milligram of protein (U/mg), where one unit corresponds to 1 μmole of 5-thio-2-nitrobenzoate produced per minute. The wild-type CAT has a specific activity of 125 U/mg.

Cytidine Deaminase.

Cytidine deaminase specific activity was determined according to published methods. The deamination of cytidine to uridine was conducted at room temperature and was determined by the rate of absorbance loss at 282 nm (Σ282=3.6 mM⁻¹ cm⁻¹). The assay was conducted in a total volume of 0.2 mL containing 0.2 mM cytidine in 50 mM Tris-HCl (pH 7.5). The reaction was initiated by the addition of about 0.3 μg/mL unpurified cytidine deaminase expressed using CFPS. Specific activity is expressed in units per milligram of protein (U/mg), where one unit corresponds to 1 mole of cytidine deaminated per minute. The wild-type E. coli cytidine deaminase has a specific activity of 250 U/mg.

Dihydrofolate reductase (DHFR).

DHFR specific activity was determined by the oxidation of NADPH (Σ340=6.22 mM⁻¹ cm⁻¹) as indicated by the decrease in absorbance at 340 nm and 30° C. The enzymatic assay was conducted in a total volume of 0.2 mL containing 45 M dihydrofolate (DHF), 60 μM NADPH, and 10 mM β-mercaptoethanol in 100 mM imidazole-chloride (pH 7.0). The reaction was initiated by addition of unpurified CFPS expressed DHFR to a final assay concentration of 0.3 μg/mL. Specific activity is expressed in units per milligram of protein (U/mg) where one unit corresponds to 1 μmole of NADP+ produced per minute. The wild-type DHFR has a specific activity of 53 U/mg.

FMN Reductase.

FMN reductase specific activity was determined by monitoring the oxidation of NADPH at room temperature and by measuring the decrease in absorbance at 340 nm (Σ340=6.2 mM⁻¹ cm⁻¹). The reaction was conducted in a total volume of 0.2 mL containing 50 mM Tris-HCl (pH 7.5), 180 μM NADPH, and 20 μM riboflavin. The reaction was initiated by the addition of unpurified FMN reductase from a CFPS reaction sample at a final concentration of about 0.3 μg/mL. Specific activity is expressed in units per milligram of protein (U/mg) where one unit corresponds to 1 μmole of NADP+ produced per minute. The wild-type FMN reductase from E. coli has a specific activity of 120 U/mg.

Glucose 6-Phosphate Dehydrogenase.

Glucose-6-phosphate dehydrogenase specific activity was determined in an assay reaction (0.2 mL) containing 100 mM Tris-HCl (pH 8.0), 10 mm MgCl₂, 0.18 mM NADP+, and 1 mM D-glucose-6-phosphate. The reaction was initiated by addition of 0.03 μg of unpurified enzyme from the CFPS reaction and the formation of reduced NADP+ (NADPH) was followed at 340 nm (Σ340=6.22 mM⁻¹ cm⁻¹). One unit of activity is the amount of enzyme required to catalyze the reduction of 1 μmole of NADP+ per min at 25 C. Specific activity is expressed in units per milligram of protein (U/mg). The wild-type glucose-6-phosphate dehydrogenase from E. coli has a specific activity of 104 U/mg.

β-Glucuronidase.

β-Glucuronidase specific activity was determined by monitoring the accumulation of the p-nitrophenyl product (pNP) arising from hydrolysis of pNP-β-D-glucuronide at room temperature and by measuring the increase in absorbance at 405 nm (Σ340=18.5 mM⁻¹ cm⁻¹). The reaction was conducted in a total volume of 0.2 mL containing 50 mM Tris-HCl (pH 7.6) and 1 mM pNP-β-D-glucuronide. The reaction was initiated by addition of 0.03 μg of unpurified β-glucuronidase from a CFPS reaction. Specific activity is expressed in units per milligram of protein (U/mg) where one unit corresponds to 1 μmole of pNP produced per minute. The wild-type GUS from E. coli has a specific activity of 60 U/mg.

β-Lactamase.

β-lactamase specific activity was determined by monitoring the hydrolysis of nitrocefin (Calbiochem, La Jolla, Calif.) at room temperature and by measuring the increase in absorbance at 486 nm (Σ486=20.5 mM⁻¹ cm⁻¹). The reaction was conducted in a total volume of 0.2 mL containing 100 mM Tris-HCl (pH 7.2) and 1 μM nitrocefin and was initiated by the addition of a CFPS reaction sample to effect a 4.000-fold dilution. No significant background activity was observed in control reactions. Specific activity is expressed in units per milligram of protein (U/mg) where one unit corresponds to 1 mole of nitrocefin hydrolyzed per minute. The wildtype β-lactamase from E. coli has a specific activity of 1,928 U/mg.

Malate Dehydrogenase.

Mdh specific activity was determined by measuring the increase in the concentration of NADH accompanying the conversion of malate to oxaloacetate (NADH, Σ340=6.2 mM⁻¹ cm⁻¹). The assay was conducted in a total volume of 0.2 mL with 0.188 mL of the assay buffer (0.168 mL of 0.1 M sodium glycinate buffer, pH 10; 0.020 mL of 1.0 M malic acid, pH 7.0) and 0.01 mL of 50 mM NAD+ (2.5 mM). The reaction was initiated by the addition of unpurified Mdh in a CFPS reaction sample to a final concentration of 0.3 μg/mL (in a volume of 2 μL). The increase in absorbance at 340 nm was monitored at room temperature. Specific activity is expressed in units per milligram of protein (U/mg) where one unit corresponds to 1 pmole of NADH produced per minute. The wild-type MDH from E. coli has a specific activity of 700 U/mg.

Thioredoxin Reductase.

Thioredoxin reductase specific activity was determined by coupling it with the enzymatic reaction of glucose-6-phosphate dehydrogenase and using the thiol reagent DTNB (Σ412=13.6 mM⁻¹ cm⁻¹). A standard assay reaction (0.2 mL) contained 200 μM DTNB, 300 μM NADPH, 0.2 U glucose-6-phosphate dehydrogenase, and 1.5 mM EDTA all in 50 mM phosphate buffer (pH 7.6). The reaction was initiated by addition of unpurified TR in a CFPS reaction sample to a final concentration of 0.3 μg/mL. Specific activity is expressed in units per milligram of protein (U/mg) where one unit corresponds to 1 μmole of 5-thio-2-nitrobenzoate produced per minute. The wild-type TR from E. coli has a specific activity of 39 U/mg.

Truncated Variant of Human Tissue Plasminogen Activator (v-tPA).

v-tPA specific activity was determined by monitoring the formation of 4-nitraniline, via the hydrolysis of N-methylsulfonyl-D-Phe-Gly-Pro-Arg-4-nitranilide acetate (ChromozymR t-PA, Roche Applied Sciences, Indianapolis, Ind.), at 405 nm and 37° C. (ε405=10.4 mM⁻¹ cm⁻¹). The reaction was conducted in a total volume of 0.11 mL containing 0.10 mL of the assay reagent mixture buffer: nine parts of Tris buffer (100 mM Tris-HCl (pH 8.5) and 0.15% (w/v) Tween 80) plus one part of Chromozym® t-PA solution (4 mM ChromozymR t-PA in ultrapure distilled water). The reaction was initiated by the addition of 10 μL of a v-tPA CFPS reaction sample. No significant background activity was observed in control reactions. Specific activity is expressed in units per milligram of protein (U/mg) where one unit corresponds to 1 μmol of 4-nitraniline produced per minute of reaction. The activity of sample was calculated by comparison with a v-tPA standard.

Uridylate Kinase.

Uridylate kinase specific activity was determined by coupling it to the enzymatic reactions of pyruvate kinase and lactate dehydrogenase, and the consumption of NADH, which was dependent on the ADP supplied by the uridylate kinase-catalyzed reaction, was measured at 340 nm and 30° C. (Σ340=6.2 mM⁻¹ cm⁻¹). The assay was conducted in a total volume of 0.2 mL containing 50 mM KCl, 2 mM MgCl₂, 1 mM phosphoenolpyruvate (PEP), 0.2 mM NADH, 1 mM ATP, 0.1 mM UMP, and 2 U each of pyruvate kinase, nucleoside diphosphate kinase, and lactate dehydrogenase in 50 mM Tris-HCl (pH 7.4). The reaction was initiated by the addition of approximately 0.3 mg/mL of unpurified uridylate kinase expressed using CFPS. Specific activity is expressed in units per milligram of protein (U/mg), where one unit is defined as the amount of enzyme required for the consumption of 1 μmol of NADH per minute. The wild-type uridylate kinase from E. coli has a specific activity of 128 U/mg.

Estimation of Translation Elongation Rate

The translation elongation rate (AAs/s) was estimated as described previously. The bulk protein accumulation rate was determined by measuring protein yields as a function of time. Twelve 15-μL sequential CFPS reactions enriched for the same effector were conducted as described above but with the addition of 4.2 μM L-[14C]-leucine at the start of the second round, and this round of expression was terminated at various time points (t=0, 10, 20, 30, 60, 90, 120, 180, 210, 240, 270, and 300 min). The amount of total reporter protein produced was estimated by measuring TCA-insoluble radioactivity using a liquid scintillation counter (Wallac 1450 Microbeta LSC, Perkin Elmer) as described above. We assumed the total protein synthesis rate was equal to the measured bulk protein accumulation rate since the protein degradation rate was negligible, as observed before. Thus, the average translation elongation rate for individual ribosomes was calculated as follows: average total protein synthesis rate (μM/s) multiplied by the number of amino acids per synthesized protein (AAs) (220 for CAT), divided by the concentration (μM) of translating ribosomes.

High-Performance Liquid Chromatography (HPLC) Analysis.

Targets that were shown to significantly influence the total reporter activities in the CFPS systems were analyzed for their effect on the metabolite composition of the reaction mixtures. The first-round expression of these effectors was initiated and reaction samples were quenched at t=0, 30, 60, 210, and 300 min as outlined below to quantify various organic acids, nucleotides, and amino acids according to previously described methods (Jewett and Swartz, 2004b) with minor modifications. Organic acids. An AgilentR 1100 series HPLC system with a G1315A diodearray detector (DAD) and a G1362A refractive index detector (RID) (Palo Alto, Calif.) was used to analyze organic acids with an AminexR HPX-87H column (Bio-Rad; Hercules, Calif.) and an isocratic separation method. CFPS reaction samples (15 μL) were quenched with equal volumes of 150 mM sulfuric acid (H2SO4), and samples were centrifuged for 10 min at 12,000×g and 4° C. to remove precipitated proteins. The supernatant was analyzed by HPLC using an injection volume of 30 μL. The column was run at 55° C. at 0.4 mL/mm for 40 min using a running buffer of 5 mM H2SO4. Compounds were identified by comparison to known standards based on retention time and their UV absorbance (210 nm) and refractive index (nRIU) signal. Calibration standards were routinely run to ensure accuracy.

Nucleotides.

An Agilen® 1100 series HPLC system (Palo Alto, Calif.) was used to separate nucleotides with a Vydac® 302IC4.6 column (Hesperia, Calif.). CFPS samples were prepared as described above for organic acid analysis. A sample of 15 μL from the supernatant was used for injection. A linear gradient separation method was employed using 10 to 125 mM sodium phosphate (adjusted to pH 2.8 with concentrated glacial acetic acid) over 30 min at a flow rate of 1.0 mL/min and at 28° C. Compounds were identified by comparison to known standards for retention time and UV absorbance (260 nm). Calibration standards were routinely run to ensure accuracy.

Amino Acids.

The Dionex AAA-Direct™ Amino Acid Analysis System (Sunnyvale. CA) was used to separate amino acids by gradient anion exchange and utilizing a gold working electrode with pulsed electrochemical detection. CFPS reaction samples were prepared as described above for organic acid analysis except that the supernatant was further diluted with water so that the initial amino acid concentration was ˜20 μM. For the quantification of arginine, the supernatant was diluted with H2SO4 to a final concentration of 0.4 M, which allowed for the separation of the arginine peak from that of the flow-through. The supernatant (20 μL) was applied to an AminoPac™ PA10 column, and the gradient elution anion exchange method was conducted according to the manufacturer's instructions. Amino acids concentrations were determined by comparison with a calibration standard. Calibration standards were routinely run to ensure accuracy.

In Vitro Transcription.

Radiolabeled CAT-mRNA transcript was obtained by the in vitro transcription of the pK7CAT plasmid with T7 RNA polymerase. The reaction mixture (50 L) contained 50 mM Tris-HCl (pH 7.5), 15 mM magnesium chloride, 5 mM dithiothreitol, 2 mM spermidine, 2 mM each of ATP, GTP, CTP, and UTP, 10 μM [³H]-UTP (Amersham GE Biosciences, Piscataway, N.J.), 1 mg/mL T7 RNA polymerase, 1 U/μL RNaseOUT RNase inhibitor (Invitrogen, Carlsbad, Calif.), and 100 μg/mL template DNA. The mixture was incubated in a humidified incubator at 37° C. for 3 hr and treated with 10 U RNase-free DNase I for 15 min at room temperature. The mRNA transcript was isolated by Trizol®/chloroform extraction according to manufacturer's instructions (Invitrogen), followed by isopropanol precipitation and ethanol wash. The pellet was immediately resuspended in RNase-free water, and the mRNA concentration and purity were measured by UV-spectroscopy using the Qubit™ fluorometer (Invitrogen). The integrity of the mRNA transcripts was analyzed by denaturing electrophoresis on 6% polyacrylamide TBE-Urea gels according to manufacturer's instructions (Invitrogen), followed by ethidium bromide staining.

mRNA Decay Assay.

The influence of effectors with known involvement in mRNA stability was analyzed by incubating in vitro transcribed-[³H]-UTP-labeled CAT mRNA transcripts in CFPS reaction mixtures enriched for the respective effectors and monitoring the degree of transcript degradation. Sequential CFPS reactions were conducted as described above with some modifications. The cell-free expression of the effectors was directed by their respective linear ETs and terminated after 40 min of incubation. Instead of the addition of a methylated plasmid template to initiate a second round of CFPS, 1.5 μM of [³H]-UTP labeled CAT-mRNA was added. The 15-μL reaction samples were quenched at various time points during the incubation period (t=0, 30, 60, 120, 240, and 300 min) to analyze the mRNA degradation rates in the presence of these effectors. The mRNA concentration and stability in the CFPS reaction mixtures were analyzed by measuring TCA-insoluble radioactivity using a liquid scintillation counter (Wallac 1450 Microbeta LSC, Perkin Elmer) and performing denaturing electrophoresis on 6% polyacrylamide TBE-Urea gels according to manufacturer's instructions (Invitrogen, Carlsbad, Calif.). The gels were dried using a gel dryer (Bio-Rad, Hercules, Calif.), exposed to phosphor screens (GE Healthcare) for 3-5 days, and scanned on a Typhoon™ Trio imager (GE Healthcare).

SDS-PAGE.

Effectors with known or presumed involvement in protein stability were analyzed for their effect on polypeptide degradation in the CFPS reaction mixtures. Sequential rounds of CFPS were conducted as described above, and reaction samples (15 μL) were quenched at various time points during the second round (t=0, 30, 60, 90, 120, 180, 240, and 300 min) to analyze the protein degradation pattern in the presence of these effectors via reducing SDS-PAGE. NuPAGE® Novex precast gels and reagents were purchased from Invitrogen (Carlsbad, Calif.). Samples (30 μg/mL) were denatured for 10 min at 70° C. in loading buffer (1×LDS running buffer and 50 mM DTT). The samples were loaded onto a 10% (w/v) Bis-Tris precast gel with SeeBlue® Plus2 molecular weight protein standard and electrophoresed in MES/SDS running buffer containing NuPAGE® antioxidant. SimplyBlue® SafeStain was used to stain and fix the gels according to the manufacturer's recommendations. The gels were dried using a gel dryer (Bio-Rad).

Validation Experiments with Commercial Enzymes.

The following enzymes were purchased to validate the effects observed in the sequential CFPS systems: AckA, GlpK, Ndk, PykF and TrxA from Sigma-Aldrich (St. Louis, Mo.); DnaJ, DnaK, GroEL, GroES, and GrpE from Enzo Life Sciences (Plymouth Meeting, Pa.); DsbG from Raybiotech (Norcross, Ga.); Fre from NovoCIB (Lyon, France); GrxA from American Diagnostica (Stamford, Conn.); and Mdh from GenWay Biotech (San Diego, Calif.). Batch CFPS reactions were conducted as described above, with the enzymes added at the beginning of the incubation period to a final concentration representing their respective yields after the first round of protein expression, as well as L-[¹⁴C]-leucine (4.2 μM). Total CAT accumulation and specific activity were determined as described above.

Statistical Analysis.

All measurements were performed in duplicate, unless indicated otherwise. Values are expressed as mean±SD from the mean. Statistical analysis was carried out using a Student's t-test.

### Results and Discussion

Development and validation of the sequential cell-free protein synthesis (CFPS) protocol. A sequential cell-free expression protocol was used to survey the entire E. coli genome to identify soluble gene products that influence in vitro transcription, translation, and protein folding (including energy metabolism and RNA and protein stability). The first round of expression was directed by transcriptionally-active linear DNA expression templates (ETs) and essentially created an array of cell extracts that were individually enriched with a single effector candidate. This round of cell-free expression was terminated by nuclease DpnII addition after 40 min of incubation. A methylated plasmid template encoding for the expression of one of the reporter enzymes was then added to initiate a second round of protein synthesis. Each first-round effector gene was evaluated for its ability to influence cell-free protein expression and/or protein folding by measuring the accumulated reporter activity and comparing to the activity in negative control (baseline) cell-free reactions. By utilizing such a platform, essentially every soluble protein was tested for its effect (direct or indirect) on the complex metabolic system.

Generation of linear DNA expression template (ET) library for genome-wide survey. The selection of the open reading frames (ORFs) evaluated in this work was based on the localization of their respective gene products in vivo. Of the 4,490 identified ORFs in the E. coli genome, only the 685 that have been identified as membrane associated were excluded from the survey. Thus, we set out to examine the effects of 3,805 ORFs, which consisted of the 914 identified as cytoplasmic; the 137 periplasmic, which were amplified without their secretion signal sequence; and the 2,754 unclassified, of which ˜35% have unknown functions or functions assigned only by homology. This set also included untranslated transcripts. Linear DNA ETs for the 3,805 gene products to be evaluated were prepared in a 96-well format. From the E. coli A19 genome sequence, gene-specific primer pairs were designed to amplify each ORF from purified genomic DNA and to introduce unique overlap regions to the gene coding sequence for subsequent extension with the transcriptional regulatory elements. Of the 3,805 ORFs targeted, 3,749 (98.5%) were successfully amplified. Under modified conditions, all of the difficult targets were amplified. The transcriptionally-active linear ETs were then generated using a two-stage, extension/amplification PCR protocol, in which the purified gene targets were extended with the T7 transcriptional regulatory elements. With this procedure, 3,637 (95.6%) of the ORF templates were successfully extended to form full length ETs.

Reaction conditions of CFPS system used for the survey. The reaction conditions of the CFPS system employed for the survey closely mimicked the cytoplasmic chemical environment, which consequently activates natural metabolism (i.e., central catabolism including glycolysis, the TCA cycle, and oxidative phosphorylation). Glutamate, which was used as the primary energy source in our in vitro expression reactions, was directed into the TCA cycle to form α-ketoglutarate by endogenous glutamate dehydrogenase (GdhA) and to produce reducing equivalents. Based on this assay system, we expected to identify effectors with several types of metabolic effects. These include energy supply, the degradation and modification of small molecular weight substrates and intermediates; degradation of DNA, RNA, and polypeptides; the stimulation or inhibition of transcription and translation; and assistance with protein folding.

For this initial study only individual effectors were examined. Anticipating that many enzymes would require cofactors for activation, we modified our cell-free expression system to include various vitamins, cofactors, and metal ions for activation of such effector candidates. In one series of analyses, the CFPS reactions were supplemented with 150 μg/mL of a molecular chaperone, inclusion body-associated protein B (IbpB). The complete survey was also conducted without the addition of the sHsp.

First-round protein expression of a subset of effector candidates. The cell-free expression of the effector gene products was directed by the purified linear ETs and conducted using 15-μL batch reactions in 96-well, flat-bottom microtiter plates. In order to conduct a comprehensive survey of the genome, three enzymes with different structural characteristics, activation requirements, and enzymatic activities were selected as reporter proteins: chloramphenicol acetyltransferase (CAT), β-lactamase, and the active domain of human tissue plasminogen activator (v-tPA). These enzymes were also chosen because they have available colorimetric assays to allow for the high-throughput evaluation of active protein accumulation in a 96-well format. The third assay protein, v-tPA, was selected to introduce a level of complexity to the metabolic analysis. This enzyme is a truncated variant of the structurally complex mammalian tPA, and requires a rather challenging folding process, which includes the formation of nine disulfide bonds that are necessary for activity. In order to ensure consistency in the survey data, all three reporter enzymes were expressed under the conditions for disulfide bond formation and both with and without IbpB addition. Therefore, each effector candidate was evaluated a total of six times.

TABLE 4
First-round expression of gene products from E. coli. The cell-free
expression of the effector gene products was directed by purified linear
DNA expression templates and conducted using 15-μL batch reactions
in 96-well, flatbottom microtiter plates; expression was terminated by
nuclease DpnII addition (1 U) after 40 min of incubation. The
accumulation of a genomic subset consisting of 96 diverse effector
candidates was evaluated by adding 4.2 μM [14C]-L-leucine into
the reaction mixtures and measuring TCA-insoluble radioactivity.
The proteins were expressed in the presence and absence of
molecular chaperone IbpB, and the average soluble protein yield of
monomers across the entire array was 6.4 ± 0.6 μM, which
was improved by ~50% with the IbpB supplementation. Results
are the average of n = 3 experiments.

| | Monomer | | Terminated | Terminated |
|---|---|---|---|---|
| | MW | Multimeric | CFPS yield of | CFPS yield w/ |
| Protein | (kDa) | state | multimer (μM) | IbpB (μM) |
| GrpE | 21.8 | monomer | 10.9 ± 0.7 | 15.5 ± 1.6 |
| GrxA | 9.7 | monomer | 31.1 ± 2.1 | 45.5 ± 4.3 |
| HchA | 31.2 | homodimer | 1.3 ± 0.2 | 1.6 ± 0.2 |
| HslO | 32.5 | monomer | 3.8 ± 0.3 | 6.3 ± 0.3 |
| HslR | 15.5 | monomer | 6.2 ± 0.4 | 8.4 ± 0.4 |
| HtpG | 71.2 | homodimer | 0.9 ± 0.1 | 1.3 ± 0.1 |
| InfA | 8.3 | monomer | 20.7 ± 0.8 | 24.3 ± 1.5 |
| InfB | 97.4 | monomer | 1.6 ± 0.1 | 2.4 ± 0.2 |
| InfC | 20.6 | monomer | 12.2 ± 0.8 | 18.3 ± 0.8 |
| LdcC | 80.6 | homodecamer | 0.1 ± 0.0 | 0.2 ± 0.0 |
| LepA | 66.6 | monomer | 1.3 ± 0.1 | 2.1 ± 0.2 |
| Lon | 87.4 | homotetramer | 0.3 ± 0.1 | 0.5 ± 0.0 |
| Lpd | 50.7 | homodimer | 2.1 ± 0.0 | 2.7 ± 0.2 |
| LysR | 34.4 | monomer | 2.5 ± 0.2 | 3.5 ± 0.1 |
| MazF | 12.1 | homodimer | 2.8 ± 0.5 | 4.2 ± 0.2 |
| MazG | 30.4 | homodimer | 2.5 ± 0.2 | 3.8 ± 0.1 |
| Mdh* | 32.3 | homodimer | 1.2 ± 0.1 | 1.5 ± 0.1 |
| MnmE | 49.2 | homodimer | 0.4 ± 0.0 | 0.6 ± 0.0 |
| NarH | 68.1 | heterodimer | 3.4 ± 0.1 | 5.4 ± 0.4 |
| Ndk | 15.5 | homotetramer | 2.4 ± 0.5 | 3.7 ± 0.2 |
| Nth | 23.6 | monomer | 4.3 ± 0.4 | 5.9 ± 0.5 |
| NusG | 20.5 | monomer | 7.3 ± 0.4 | 10.4 ± 0.6 |
| PcnB | 53.9 | monomer | 1.4 ± 0.1 | 2.2 ± 0.1 |
| Pnp | 77.1 | homotrimer | 0.7 ± 0.1 | 0.9 ± 0.0 |
| PrfA | 40.5 | monomer | 1.2 ± 0.1 | 1.9 ± 0.2 |
| PrfB | 41.3 | monomer | 1.7 ± 0.2 | 2.6 ± 0.2 |
| PyrH* | 26.0 | homohexamer | 0.9 ± 0.1 | 1.4 ± 0.1 |
| Pth | 21.0 | monomer | 5.3 ± 0.2 | 8.4 ± 0.5 |
| RaiA | 12.8 | monomer | 17.1 ± 1.5 | 24.6 ± 2.4 |
| Rna | 29.6 | monomer | 5.6 ± 0.2 | 7.7 ± 0.5 |
| Rnb | 72.5 | monomer | 1.8 ± 0.1 | 2.6 ± 0.1 |
| Rne | 118.2 | homotetramer | 0.2 ± 0.0 | 0.2 ± 0.0 |
| Rng | 55.4 | homodimer | 0.7 ± 0.1 | 1.1 ± 0.1 |
| Rnr | 92.1 | monomer | 1.1 ± 0.1 | 1.6 ± 0.1 |
| RpsP | 9.2 | monomer | 11.1 ± 0.9 | 14.9 ± 1.1 |
| RraA | 17.4 | homotrimer | 2.6 ± 0.4 | 3.7 ± 0.1 |
| RraB | 15.6 | monomer | 15.7 ± 1.3 | 26.4 ± 1.9 |
| SdaB | 48.8 | monomer | 2.4 ± 0.1 | 3.5 ± 0.3 |
| SpoT | 79.3 | monomer | 1.7 ± 0.1 | 2.5 ± 0.2 |
| TdcB | 35.2 | homotetramer | 0.4 ± 0.1 | 0.5 ± 0.0 |
| TdcG | 48.5 | homodimer | 1.4 ± 0.2 | 2.2 ± 0.2 |
| Tdh | 37.2 | homotetramer | 0.7 ± 0.1 | 1.4 ± 0.1 |
| AbgA | 48.6 | monomer | 1.8 ± 0.1 | 2.3 ± 0.1 |
| AceE | 99.7 | homodimer | 0.4 ± 0.0 | 0.7 ± 0.1 |
| AceF | 66.1 | monomer | 1.6 ± 0.1 | 2.4 ± 0.2 |
| AckA | 43.3 | monomer | 6.4 ± 0.4 | 8.5 ± 0.3 |
| AdiA | 84.4 | homodecamer | 0.2 ± 0.0 | 0.3 ± 0.0 |
| AnsA | 37.1 | homodimer | 2.1 ± 0.1 | 3.1 ± 0.3 |
| ArgR | 17.0 | homohexamer | 2.4 ± 0.6 | 3.8 ± 0.2 |
| CadA | 81.3 | homodecamer | 0.1 ± 0.0 | 0.2 ± 0.0 |
| Cdd* | 31.5 | homodimer | 5.5 ± 0.8 | 7.6 ± 0.7 |
| CspA | 7.4 | monomer | 9.9 ± 1.1 | 14.2 ± 0.6 |
| CspB | 7.7 | monomer | 12.2 ± 1.1 | 18.4 ± 1.6 |
| CspE | 7.5 | monomer | 11.3 ± 1.3 | 16.1 ± 0.3 |
| DegP | 49.4 | homohexamer | 0.5 ± 0.1 | 0.7 ± 0.0 |
| DegQ | 47.2 | monomer | 2.7 ± 0.1 | 3.1 ± 0.2 |
| DeoA | 47.2 | homodimer | 2.2 ± 0.3 | 2.6 ± 0.1 |
| DnaJ | 41.1 | monomer | 2.6 ± 0.2 | 3.7 ± 0.3 |
| DnaK | 69.1 | monomer | 1.9 ± 0.1 | 2.7 ± 0.1 |
| DsbA | 23.1 | monomer | 7.7 ± 0.3 | 11.4 ± 0.9 |
| DsbG | 29.8 | homodimer | 2.5 ± 0.1 | 4.2 ± 0.2 |
| EndA | 26.7 | monomer | 3.4 ± 0.4 | 4.9 ± 0.2 |
| FolA* | 18.0 | monomer | 6.7 ± 0.5 | 8.2 ± 0.7 |
| Fre* | 26.2 | monomer | 5.2 ± 0.3 | 7.2 ± 0.4 |
| Frr | 23.5 | monomer | 3.5 ± 0.2 | 5.4 ± 0.2 |
| FtsZ | 40.3 | monomer | 3.5 ± 0.1 | 4.8 ± 0.4 |
| FtsL | 13.6 | monomer | 5.5 ± 0.4 | 8.2 ± 0.3 |
| FtsW | 46.0 | monomer | 1.8 ± 0.1 | 2.3 ± 0.1 |
| FusA | 77.6 | monomer | 1.7 ± 0.1 | 2.3 ± 0.1 |
| GcvR | 20.8 | monomer | 9.2 ± 0.6 | 11.3 ± 0.3 |
| GreA | 17.6 | monomer | 10.6 ± 0.4 | 13.6 ± 0.5 |
| GroEL | 57.3 | homo-14-mer | 0.1 ± 0.0 | 0.2 ± 0.0 |
| GroES | 10.4 | homoheptamer | 3.6 ± 2.1 | 5.1 ± 0.4 |

*Activity confirmed using established colorimetric enzymatic activity assays (see Materials and Methods)

The activities of several enzymes were evaluated with available colorimetric assays. The specific activities of all of the tested proteins suggested that greater than 50% of the produced protein was active, which was consistent with our previous work. In the presence of IbpB, the specific activities of these enzymes improved by an average of ˜20%. Results are the average of n=3 experiments.

TABLE 5

| PROTEIN | | | | |
|---|---|---|---|---|
| wild-type specific activity* | Standard CFPS | | CFPS w/IbpB | |
| U/mg (reference) | soluble (μM) | active* (%) | soluble (μM) | active* (%) |
| UMP kinase | 0.9 ± 0.1 | 52 ± 10 | 1.4 ± 0.1 | 63 ± 9 |
| 128 U/mg (Bucurenci et al., | | | | |
| 1998) | | | | |
| Dihyrofolate reductase | 6.7 ± 0.5 | 81 ± 12 | 8.2 ± 0.7 | 93 ± 9 |
| 53 U/mg (Mouat, 2000) | | | | |
| Thioredoxin reductase | 2.3 ± 0.2 | 83 ± 5 | 2.9 ± 0.2 | 90 ± 4 |
| 39 U/mg (Knapp and Swartz, | | | | |
| 2004) | | | | |
| Cytidine deaminase | 5.5 ± 0.8 | 68 ± 14 | 7.6 ± 0.7 | 73 ± 7 |
| 250 U/mg (Yang et al., 1992) | | | | |
| Malate dehydrogenase | 1.2 ± 0.1 | 51 ± 2 | 1.6 ± 0.1 | 62 ± 7 |
| 700 U/mg (Breiter et al., 1994) | | | | |
| Glucose 6-phosphate | 1.1 ± 0.1 | 62 ± 3 | 1.8 ± 0.1 | 86 ± 12 |
| dehydrogenase | | | | |
| 104 U/mg (Banerjee and | | | | |
| Frankel, 1972) | | | | |
| FMN reductase | 5.2 ± 0.3 | 79 ± 9 | 7.2 ± 0.4 | 85 ± 2 |
| 120 U/mg (Fieschi et al., 1995) | | | | |
| β-Glucuronidase | 1.4 ± 0.4 | 81 ± 10 | 2.2 ± 0.2 | 90 ± 8 |
| 60 U/mg (Geddie and | | | | |
| Matsumura, 2004) | | | | |

Several gene products with known effects were assessed to validate the protocol: elongation factors EF-Tu and EF-Ts; nucleoside diphosphate kinase (Ndk); ribonuclease E (RNase E); glycerol kinase (GlpK); and arginine decarboxylase (SpeA). These proteins exerted the expected effects on the three reporters, with and without IbpB. These effector proteins were used as on-plate positive controls during the genomic survey.

On-plate negative control (i.e., baseline) reactions were also conducted in parallel with the effector-enriched CFPS reactions during the survey. Each first-round effector gene product was evaluated for its ability to influence cell-free protein expression and/or protein folding by measuring the accumulated reporter activity and comparing to the activity in these control reactions. For comparison, baseline activities for each of the reporter proteins were measured after the first-round expression of two orthogonal proteins: human granulocyte macrophage stimulating factor (hGM-CSF) fused to a single-chain antibody fragment (V20-VLVH) and the V20-VLVH scFv alone. Changes from these baseline values were then used to identify positive and negative effectors which significantly influenced accumulation of the active reporters. Significant effects were defined as those exceeding the standard deviation of the negative control reactions on a given day.

Based on the many metabolic activities listed in Table 1, we expected to identify at least 100 gene products that significantly affected the cell-free metabolism. In fact, we identified 139 proteins that exhibited significant influence on the system: 79 proteins enhanced the total enzyme production (total enzyme activity) (i.e., positive effectors), while 60 reduced this quantity (i.e., negative effectors). As we had anticipated, this set of effectors represented proteins with vastly divergent metabolic roles, including involvement in energy supply; mRNA synthesis; RNA, nucleotide, and amino acid stability; and translation initiation and elongation rates as well as protein folding, activation, and stability. Of these effectors, 133 had similar effects on the three reporter proteins. The 6 effectors that exhibited different influences on the reporters were periplasmic chaperone Skp; periplasmic proteases DegP, DegQ, and PtrA; thioredoxin reductase TrxB; and glutathione reductase Gor, Skp and DegP enhanced the accumulated activity of only PJ-actamase by 34 and 31%, respectively; while, DegQ and PtrA reduced β-lactamase activity by 12 and 11%, respectively. The fact that these effectors influenced the activity of only the periplasmic reporter protein was consistent with their functions. The enrichment of the CFPS reactions for TrxB and Gor led to 31 and 43% reductions in accumulated v-tPA activity, respectively; while these same TrxB and Gor-enrichments enhanced CAT and β-lactamase activities by 29 and 26%, respectively. We believe that the different effects of TrxB and Gor on the activities of the reporter proteins were due to the effectors' roles in both disulfide bond reduction and general protein folding.

Furthermore, 92.1% of the effectors (128 effectors) were identified both with and without IbpB supplementation and exhibited similar effects under the different conditions; whereas. 11 of the effectors were identified only in the presence of IbpB. This fact indicates that only 7.9% of the identified gene products were expressed at low active yields in the standard CFPS system and required additional folding assistance (Table 4B). Unsurprisingly, many of these low-expressing effectors were high molecular weight, multimeric gene products.

Determining whether the effectors influenced protein accumulation or specific activity. We evaluated a subset of 115 of the most significant identified effectors. We conducted sequential rounds of CFPS to determine whether these proteins influenced the in vitro expression system by affecting either protein accumulation or protein folding by measuring the percent change from baseline concentration and baseline specific activity. We studied the activities of these effectors in CAT-expressing CFPS reactions with and without IbpB supplementation. The classification of each protein as an effector of either CAT accumulation or CAT folding, based on the observed effects on the CFPS system, was consistent with the known or predicted in vivo roles of the gene product. Many of the proteins identified as effectors of cell-free expression have been observed or presumed to influence the intracellular metabolic system, mainly at the transcriptional and translational levels, in manners that would consequently affect protein accumulation. For example, transcription factors (Csps A, B, C, E, F, and G) and translation factors (IF 1-3, EFs Tu, Ts, G, and P) were identified as such effectors. Also included in this set were various gene products involved in the energy supply (AckA, GdhA, GlpK, Ndk, PykF, and YdaM); RNA stability (ribonucleases and mRNA interferase toxins); small molecule substrate supply (MazG, YhcM, YjdA, and EutP); and protein stability (PtrA and YpdF). The gene products identified as effectors of protein activity in our CFPS system were a variety of molecular chaperones, whose key or secondary roles in vivo include assisting and/or catalyzing the proper folding of macromolecules. Represented in this group of effectors were some of the major Hsp families (Hsp10 (GroES), Hsp60 (GroEL), Hsp700 (DnaK, HscA, and HscC), and Hsp100 (ClpA and ClpB)); Dsb oxidoreductases and reductases; and sulfhydryl-disulfide redox-modulating factors (TrxA, TrxB, GrxB, and Gor).

TABLE 6A
Effectors of accumulated reporter activities identified using a sequential CFPS protocol. First-
round expression of effector candidate proteins was terminated after a 40-min incubation period
by the addition of 1 U of DpnII, and reporter protein expression was initiated by adding a
methylated plasmid template. Total reporter activity was measured at the end of the reaction
and was compared to the baseline (on-plate negative control) value. 3,789 gene products of E. coli
were evaluated under 6 different CFPS conditions: 3 reporter enzymes (CAT, β-lactamase,
and v-tPA), each expressed with and without molecular chaperone IbpB, and each CFPS
reaction was conducted in duplicate. 139 gene products significantly affected the activities of the
reporter proteins (79 positively and 60 negatively) and had vastly different metabolic functions
and roles, 95.7% had similar effects on the three reporter proteins, and 92.1% of all the
effectors were identified with and without IbpB supplementation. Significant effects were defined
as those exceeding the standard deviation of the negative control reactions on a given day, and
most results are the average of n = 12 experiments, except for those identified only with IbpB
supplementation, for which n = 6 experiments.

| Metabolic Function or Role of | % Change from | Metabolic Function or Role of | % Change from |
|---|---|---|---|
| Effector | Baseline Activity | Effector | Baseline Activity |
| Energy (ATP and GTP) supply | | Amino acid supply | |
| Acetate kinase AckA | 74.6 ± 10.1 | Threonine dehydrogenase Tdh* | −13.2 ± 1.1 |
| Lipoamide dehydrogenase Lpd | 52.4 ± 4.2 | Aspartate kinase ThrA* | −14.7 ± 1.4 |
| Nucleoside diphosphate kinase Ndk | 52.2 ± 6.9 | Aspartate kinase MetL | −17.2 ± 1.3 |
| FMN reductase Fre | 44.4 ± 2.6 | Glutaminase YnaH | −18.8 ± 2.5 |
| Pyruvate kinase PykF | 42.6 ± 5.4 | Threonine dehydratase TdcB* | −19.7 ± 1.6 |
| Glutamate dehydrogenase GdhA | 26.6 ± 3.3 | Glutaminase YbaS* | −23.1 ± 1.5 |
| G6P dehydrogenase Zwt | 15.9 ± 2.2 | Tryptophanase TnaA* | −26.3 ± 1.2 |
| Diguanylate cyclase YdaM | −13.4 ± 1.8 | Lysine decarboxylase CadA* | −27.3 ± 2.8 |
| Diguanylate cyclase YddV | −24.3 ± 2.6 | Serine deaminase TdcG | −27.4 ± 2.4 |
| Malate dehydrogenase Mdh | −27.4 ± 2.4 | Arginine decarboxylase AdiA* | −27.7 ± 2.2 |
| Diguanylate cyclase YneF | −27.6 ± 3.2 | Serine deaminase SdaB | −28.6 ± 2.6 |
| Glycerol kinase GlpK | −98.5 ± 10.4 | Glutamate-cysteine ligase GshA | −29.2 ± 4.2 |
| NTP (UTP, CTP, GTP, ATP) supply | | Arginine decarboxylase SpaA | −30.4 ± 1.5 |
| Cytidine deaminase Cdd | 68.8 ± 5.5 | Lysine decarboxylase LdcC* | −31.2 ± 2.7 |
| Uridylate kinase PyrH | 18.3 ± 1.1 | Serine ammonia-lyase SdaA | −33.4 ± 2.1 |
| Protein w/ NTPase domain YjdA | −11.4 ± 0.7 | Aspartate kinase LysC | −36.8 ± 5.2 |
| Metabolic Function or Role of | % Change from | Metabolic Function or Role of | % Change from |
| Effector | Baseline Activity | Effector | Baseline Activity |
| NTP (UTP, CTP, GTP, ATP) supply (cont.) | | Transcription | |
| Protein w/ NTPase domain EutP | −12.6 ± 1.7 | Elongation factor GreB | 28.4 ± 2.2 |
| Protein with NTPase domain YhcM | −15.7 ± 1.4 | Elongation factor GreA | 23.6 ± 1.4 |
| NTP pyrophosphohydrolase MazG | −32.8 ± 2.4 | Termination factor NusG | 11.2 ± 1.3 |
| RNA stability | | Transcriptional regulator AsnC | −12.7 ± 0.7 |
| Ribonuclease E inhibitor RraB | 34.6 ± 4.4 | Predicted regulation GcvR | −13.3 ± 1.9 |
| Ribonuclease E inhibitor RraA* | 28.6 ± 3.1 | Transcriptional regulator TyrR | −13.9 ± 1.1 |
| Poly(A) polymerase PonB | 9.6 ± 0.7 | Transcriptional repressor TrpR | −14.4 ± 0.8 |
| Ribonuclease I RNase I | −12.6 ± 0.7 | Transcriptional regulator Lrp | −15.6 ± 1.2 |
| Toxin YafQ | −12.8 ± 0.9 | Transcriptional regulator LysR | −18.3 ± 2.6 |
| Toxin YafO | −14.6 ± 1.9 | Transcriptional activator ArgP | −19.6 ± 2.6 |
| Ribonuclease R RNase R | −19.9 ± 1.6 | Transcriptional regulator ArgR* | −34.2 ± 4.9 |
| Toxin YceB | −28.6 ± 1.2 | Translation | |
| Ribonuclease III RNase III | −24.4 ± 1.9 | Elongation factor EF-Tu | 61.6 ± 3.6 |
| Toxin SymE | −26.6 ± 1.8 | Ribosome recycling factor Prr | 59.4 ± 4.3 |
| Ribonuclease G RNase G | −27.4 ± 3.9 | (p)ppGpp synthetase SpoT | 47.7 ± 3.9 |
| Toxin YpjF | −27.6 ± 2.2 | Elongation factor EF-P | 46.6 ± 6.7 |
| Toxin Ykft | −29.8 ± 2.4 | Heat-shock protein HsfR | 44.9 ± 3.6 |
| Toxin YeeV | −31.2 ± 1.9 | Elongation factor 4 LepA | 36.7 ± 2.8 |
| Polynucleotide phosphorylase Pnp | −31.7 ± 2.1 | Elongation factor EF-G | 31.2 ± 2.7 |
| Toxin RelE | −39.6 ± 3.4 | Release factor RF-2 | 29.6 ± 2.6 |
| Ribonuclease II RNase II | −48.4 ± 3.7 | Predicted elongation factor YigZ | 24.3 ± 3.4 |
| Toxin MazF | −54.6 ± 3.4 | Release Factor RF-1 | 24.3 ± 1.2 |
| Ribonuclease E RNase E | −76.4 ± 5.7 | Putative GTP-binding protein YchF | 18.7 ± 1.3 |
| | | Elongation factor EF-Ts | 17.6 ± 2.3 |
| | | Initiation factor IF-2 | 17.5 ± 1.9 |
| | | Initiation factor IF-1 | 15.6 ± 1.3 |
| Metabolic Function or Role of | % Change from | Metabolic Function or Role of | % Change from |
| Effector | Baseline Activity | Effector | Baseline Activity |
| Transcription and translation | | Translation (cont.) | |
| Cold-shock protein CspA | 56.3 ± 7.2 | Release factor RF-3 | 15.2 ± 0.9 |
| Cold-shock protein CspE | 32.2 ± 1.7 | (p)ppGpp synthetase RelA | 13.7 ± 1.9 |
| Cold-shock protein CspB | 26.4 ± 3.4 | Initiation factor YciH | 11.4 ± 1.7 |
| Cold-shock protein CspC | 20.4 ± 1.3 | Initiation factor IF-3 | 10.3 ± 0.4 |
| Cold-shock protein CspG | 15.4 ± 1.1 | Ribosome stability factor RaiA | −29.3 ± 2.6 |
| Cold-shock protein CspF | 15.3 ± 1.2 | Peptidyl-tRNA hydrolase Pth | −31.2 ± 3.8 |
| Protein folding | | Transfer-messenger RNA tmRNA | −41.3 ± 5.7 |
| 60 kDa chaperonin GroEl. | 92.3 ± 13.7 | Protein folding (cont.) | |
| Disulfide isomerase DsbC | 60.4 ± 3.6 | Thioredoxin homologue YbbN | 29.7 ± 2.1 |
| sHsp IbpB | 56.2 ± 3.9 | Predicted Hsp YgeG | 29.4 ± 2.7 |
| 10 kDa chaperonin GroES | 34.6 ± 5.8 | Thioredoxin reductase TrxB | 28.9 ± 3.7 |
| Hsp70 DnaK | 53.2 ± 4.7 | (for CAT & β-lactamase) | |
| Thioredoxin TrxA | 44.6 ± 5.2 | Hsp70 homologue HscA | 27.6 ± 1.5 |
| sHsp IbpA | 41.8 ± 6.1 | Predicted Hsp YciM | 26.8 ± 3.4 |
| Hsp40 DnaJ | 37.6 ± 5.4 | Glutathione reductase Gor | 25.6 ± 1.6 |
| Hsp33 HslO | 34.7 ± 2.5 | (for CAT & β-lactamase) | |
| Periplasmic chaperone Skp | 33.6 ± 2.9 | Glutaredoxin GrxA | 23.3 ± 1.7 |
| (for β-lactamase) | | Hsp31 HchA | 23.2 ± 1.8 |
| Disulfide oxidoredutase DsbG | 33.4 ± 2.3 | Co-chaperone of HspC DjiC | 22.9 ± 1.1 |
| Predicted Hsp YcaL | 33.1 ± 4.7 | ATP-dependent protease Lon | 22.1 ± 1.8 |
| Hsp90 homologue HtpG | 31.4 ± 4.3 | Disulfide oxidoredutase DsbA | 21.2 ± 1.2 |
| Serine protease DegP (for β-lactamase) | 31.2 ± 3.3 | Hsp20 GrpE | 16.4 ± 1.1 |
| | | Hsp100 ClpB* | 15.4 ± 2.5 |
| | | Co-chaperone of HscA HscB | 14.6 ± 1.3 |
| Metabolic Function or Role of | % Change from | Metabolic Function or Role of | % Change from |
| Effector | Baseline Activity | Effector | Baseline Activity |
| Protein folding (cont.) | | Unexplained influences | |
| DnaJ homologue CbpA | 13.2 ± 2.1 | β-Glucunonidase UldA | 47.9 ± 3.9 |
| Hsp100 ClpA | 11.7 ± 0.8 | Signal recognition particle protein Flh | 40.7 ± 2.5 |
| Hsp70 homologue HscC | 11.4 ± 0.6 | Endonuclease III XthA | 36.9 ± 2.5 |
| DnaJ homologue DjlA | 11.3 ± 1.1 | Essential cell division protein FtsL | 33.6 ± 2.9 |
| Thioredoxin reductase TrxB (for v-tPA) | −31.2 ± 2.2 | Thymidine phosphorylase DsoA | 30.2 ± 2.3 |
| Glutathione reductase Gor (for v-tPA) | −43.3 ± 5.7 | Essential cell division protein FtsW | 27.6 ± 1.7 |
| Protein stability | | Essential cell division protein FtsZ | 21.8 ± 1.6 |
| Periplasmic protease PtrA | −10.7 ± 0.6 | Asparaginase AnsB | 20.6 ± 2.9 |
| (for β-lactamase) | | Asparaginase AnsA | 17.4 ± 1.1 |
| Periplasmic protease DegO | 12.2 ± 1.8 | Methionine aminopeptidase Map | 12.3 ± 0.8 |
| (for β-lactamase) | | Dihydrofolate reductase FolA | −17.4 ± 1.3 |
| Aminopeptidase YpdF | −23.8 ± 1.6 | Dihydrolipoamide acetyltransferase AceF | −45.4 ± 3.7 |
| Aminopeptidase PepN | −29.7 ± 4.2 | | |

*identified only with IbpB supplementation

TABLE 6B
Effectors of accumulated reporter activities identified only when IbpB was present in the
sequential CFPS reactions.

| | | | Terminated | Terminated | |
|---|---|---|---|---|---|
| | Monomer MW | Multimeric | CFPS yield of | CFPS yield w/ | % Change from |
| Effector | (kDa) | state | multimer (μM) | IbpB (μM) | baseline activity |
| Arginine | 84.4 | homodecamer | 0.2 ± 0.0 | 0.3 ± 0.0 | −27.7 ± 2.2 |
| decarboxylase | | | | | |
| AdiA | | | | | |
| Aspartate | 89.1 | homotetramer | 0.1 ± 0.1 | 0.2 ± 0.0 | −14.7 ± 1.4 |
| kinase ThrA | | | | | |
| Glutaminase | 32.9 | homotetramer | 1.6 ± 0.3 | 2.5 ± 0.2 | −23.1 ± 1.5 |
| YbaS | | | | | |
| Hsp100 ClpB | 95.6 | homohexamer | 0.8 ± 0.1 | 1.4 ± 0.2 | 15.4 ± 2.5 |
| Lysine | 81.3 | homodecamer | 0.1 ± 0.1 | 0.2 ± 0.0 | −27.3 ± 2.8 |
| decarboxylase | | | | | |
| CadA | | | | | |
| Lysine | 80.6 | homodecamer | 0.1 ± 0.1 | 0.2 ± 0.0 | −31.2 ± 2.7 |
| decarboxylase | | | | | |
| LdcC | | | | | |
| Ribonuclease E | 17.4 | homotrimer | 2.6 ± 0.4 | 3.7 ± 0.1 | 28.6 ± 3.1 |
| inhibitor RraA | | | | | |
| Threonine | 35.2 | homotetramer | 0.4 ± 0.1 | 0.5 ± 0.0 | −19.7 ± 1.6 |
| dehydratase | | | | | |
| TdcB | | | | | |
| Threonine | 37.2 | homoletramer | 0.7 ± 0.2 | 1.4 ± 0.1 | −13.2 ± 1.1 |
| dehydrogenase | | | | | |
| Tdh | | | | | |
| Transcriptional | 17.0 | homohexamer | 2.4 ± 0.6 | 3.8 ± 0.2 | −34.2 ± 4.9 |
| regulator ArgR | | | | | |
| Tryptophanase | 52.8 | homotetramer | 1.7 ± 0.2 | 2.2 ± 0.2 | −26.3 ± 1.2 |
| TnaA | | | | | |

TABLE 7
Identification of gene products as effectors of either CAT expression or CAT
specific activity. A subset of 115 effectors with known or predicted involvement
in either protein expression or protein folding or with unexplained influences
was analyzed to determine whether these products influenced the in vitro
expression system in a manner similar to their assigned in vivo functions.
Each gene product was evaluated for its ability to affect either CAT
accumulation or CAT specific activity, and the observed influence of each
protein was consistent with its known or predicted function.

| Factors affecting CAT expression | Factors affecting CAT specific activity |
|---|---|
| Acetate kinase AckA | 10 kDa chaperonin GroES |
| Aminopeptidases PepN, YpdF | 60 kDa chaperonin GroEL |
| Asparaginases AnsA, AnsB | ATP-dependent protease Lon |
| Colo-shock proteins Csp A, B, C, E, F, G | Co-chaperone of HscA HscB |
| Conserved proteins EulP, YhcM, YjdA | Co-chaperone of HscC DjiC |
| Cytidine deaminase Cdd | Disulfide isomerase DsbC |
| Diguanylate cyclases YdaM, YddV, YneF | Disulfide oxidoreductase DsbA |
| Dihydrofolate reductase FolA | Disulfide oxidoreductase DsbG |
| Dihydrolipoamide acetyltransferase AceF | DnaJ homologues CbpA, DjiA |
| Elongation factors EF-G, P, Ts, Tu, YigZ | Elongation factor 4 LepA |
| Transcription factors GreA, GreB, NusG | Glutaredoxin GrxA |
| Endonuclease III XthA | Glutathione reductase Gor |
| Essential cell division proteins FtsL, FtsW, FtsZ | Hsps 100 ClpA, ClpB |
| FMN reductase Fre | Hsp20 GrpE |
| G6P dehydrogenase Zwf | Hsp31 HchA |
| β-Glucuronidase UidA | Hsp33 HslO |
| Glutamte dehydrogenase GdhA | Hsp40 DnaJ |
| Glycerol kinase GlpK | Hsp70 DnaK |
| Heat-shock protein HslR | Hsp70 homologues HscA, HscC |
| Initiation factors IF-1, IF-2, IF-3, YciH | Hsp90 homologue HtpG |
| Lipoamide dehyrogenase Lpd | Predicted Hsps YcaL, YciM, YgeG |
| Malate dehydrogenase Mdh | sHsps IbpA, IbpB |
| Methionine aminopeptidase Map | Signal recognition particle protein Ffh |
| NTP pyrophosphohydrolase MazG | Thioredoxin homologue YbbN |
| Nucleoside diphosphate kinase Ndk | Thioredoxin reductase TrxB |
| Peptidyl-tRNA hydrolase Pth | Thioredoxin TrxA |
| Poly(A) polymerase PcnB | |
| Polynucleotide phosphorylase Pnp | |
| (p)ppGpp synthetases RelA, SpoT | |
| Putative GTP-binding protein YchF | |
| Pyruvate kinase PykF | |
| Release factors RF-1, RF-2, RF-3 | |
| Ribonucleases I, II, III, E, G, R | |
| Ribonuclease E inhibitors RraA, RraB | |
| Ribosome recycling factor Frr | |
| Ribosome stability factor RalA | |
| sHsps IbpA, IbpB | |
| Thymidine phosphorylase DeoA | |
| Toxins MazF, RelE, SymE, YafO, YafQ | |
| Toxins YeeV, YkfI, YoeB, YpjF | |
| Transfer-messenger RNA tmRNA | |
| Uridylate kinase PyrH | |

Black text = positive effectors
Red text = negative effectors

Validation of system responses to positive and negative effectors. The activities of some of the positive and negative effectors were compared against those of purified proteins. Commercially available proteins were purchased and added to the CFPS reaction mixtures at concentrations representing their respective yields after the first round of protein expression. The effects of the following proteins were analyzed: Ndk, AckA, PykF, GlpK, Mdh, Fre, GroES, GroEL, DnaK, DnaJ, GrpE, TrxA, GrxA, and DsbG. The expression of CAT was conducted in batch CFPS reactions as described previously, and total accumulation and specific activity of the reporter protein were measured and compared to the baseline values. The effects of the commercial products on protein production and activity were consistent with those observed in the genome-wide survey.

E. coli gene products identified as effectors of in vitro protein expression and protein folding. As expected, we identified gene products that influenced nearly every aspect of our CFPS system. The effectors were categorized as follows: (1) Factors affecting the energy (ATP and GTP) supply (for transcription, translation, and protein folding processes) (11 effectors); (2) Factors affecting the supply of nucleotide (NTPs) and amino acids for polymerization reactions (i) Nucleotide (NTP) supply (for energy and transcription) (6 effectors); (ii) Amino acid supply (for translation) (16 effectors); (3) Factors affecting transcription (11 effectors); (4) Factors affecting RNA stability (19 effectors); (5) Factors affecting translation (21 effectors); (6) Factors affecting both transcription and translation (6 effectors); (7) Factors affecting protein folding (32 effectors); (8) Factors affecting protein stability (4 effectors); and (9) Factors with unexplained influences (13 effectors).

Most of the observed effects were consistent with the in vivo metabolic functions of the gene products. Many other observations were not anticipated or as easily understood. We conducted various subsequent assays (e.g., analysis of amino acid, nucleotide, and central metabolite levels; mRNA and protein stability; and polypeptide synthesis rates) and in depth literature searches in order to generate and, in some cases, test hypotheses for the in vitro activity of the identified effectors.

Factors affecting the energy (ATP and GTP) supply. Several proteins influenced ATP and GTP regeneration in our CFPS system. This class of effector proteins both enhanced and limited the supply of energy in the CFPS system (i.e., positive and negative effectors, respectively). We identified three gene products that directly affected the production rate of reducing equivalents: positive effectors glutamate dehydrogenase (GdhA, +27%) and glucose 6-phosphate dehydrogenase (Zwf, +16%); and the negative effector malate dehydrogenase (Mdh, −27%). There were two effectors that increased the overall flux of metabolic intermediates toward substrate-level ATP production: positive effectors acetate kinase (AckA, +75%) and pyruvate kinase (PykF, +43%). We also identified two enzymes that apparently increased the transfer of electrons to the ETC for ATP generation: positive effectors FMN reductase (Fre, +44%) and the lipoamide dehydrogenase (Lpd, +52%) component of the pyruvate dehydrogenase (PDH) multi-enzyme complex. Finally, five of the identified effectors exhibited enzymatic activities that either increased or depleted the concentrations of the energy carriers ATP and GTP in the CFPS reactions: positive effector nucleoside diphosphate kinase (Ndk, +52%); and negative effectors diguanylate cyclases YdaM (−13%), YddV (−24%), and YneF (−28%); and glycerol kinase (GlpK, −99%).

Effectors that affected production rate of reducing equivalents. The first-round expression of three gene products most likely influenced the energy supply and, consequently, production performance in the CFPS system because of their roles in the generation or consumption of reducing equivalents (NADH and NADPH). The enrichment of the CFPS reactions for the positive effectors GdhA and Zwf improved CAT production by 25 and 16%, respectively; while negative effector Mdh led to a 29%-reduction in total CAT yields.

Factors affecting transcription. Since the transcription and translation processes are combined in our cell-free expression system, the efficient generation of mRNA transcripts is critical to the overall productivity of the system. We identified eleven proteins that presumably affected the transcription process in the CFPS reactions: negative effectors AsnC (−13%), GcvR (−13%), TyrR (−14%), TrpR (−14%), Lrp (−16%), LysR (−18%), ArgP (−20%), and ArgR (−34%); and positive effectors NusG (+16%), GreA (+25%), and GreB (+27%). Two factors that greatly affect transcription are (1) the accessibility of the DNA template to the RNA polymerase (RNAP) and essential transcription factors; and (2) the stability of expression template in the system. In our survey, we identified eight gene products that presumably restricted access to the expression plasmids for the reporters by binding to the DNA, and one of the eight effectors also compromised the DNA stability. These DNA-binding effectors are AsnC, GcvR, TyrR, TrpR, Lrp, LysR, ArgP, and ArgR, and their overexpression in the CFPS reactions decreased the accumulated enzymatic activities of the reporters.

Ribonucleases. The major exoribonucleolytic and endoribonucleolytic activities in E. coli were observed to significantly decrease the accumulated activities of the reporter proteins. These included the exoribonucleases RNase II, PNPase, and RNase R; and the endoribonucleases RNase E, RNase G. RNase III, and RNase I. The exoribonucleases RNase II, PNPase, and RNase R had negative effects, reducing total CAT accumulation by 46, 33, and 21%, respectively. In vivo, ribonuclease II (RNase II), encoded by the rnb gene, and polynucleotide phosphorylase (PNPase), encoded by the pnp gene, play a significant role in the decay of single-stranded mRNA by processively degrading the nucleic acid in the 3′ to 5′ direction. RNase II hydrolyzes mRNA to ribonucleoside 5′-monophosphates whereas PNPase catalyzes the phosphorolytic reaction, thus generating ribonucleoside 5′-diphosphates. Ribonuclease R(RNase R, −21%) is also a processive 3′ to 5′ exoribonuclease and is closely related to RNase II, in terms of its catalytic properties. In addition, endoribonucleases RNase E (˜74%), RNase G (˜30%), RNase III (˜20%), and RNase I (˜17%) decreased the total CAT yields. Ribonuclease E (RNase E, −74%), encoded by rne, is the major single-strand specific endonuclease in E. coli and exhibited the second-greatest negative effect on CFPS performance. Ribonuclease G (RNase G, −30%) is involved in the maturation of the 5′ end of 16S rRNA and in mRNA turnover. Ribonuclease III (RNase III, −20%), encoded by the rnc gene, digests double stranded RNA. Ribonuclease I (RNase I, −17%), encoded by the ma gene, is a periplasmic nuclease with broad substrate specificity, but is largely responsible for the degradation of total and ribosomal RNA during both normal and nutrient starvation conditions.

mRNA stabilizers. We identified three proteins that improved the stability of the mRNA transcripts, which consequently enhanced the expression of reporter CAT: RraB (+33%), RraA (+30%), and PcnB (+13%). Two inhibitors of RNase E (RraB and RraA) were identified as positive effectors and had a significant influence on the CFPS performance most likely due to the strong negative effect of endogenous levels of RNase E on in vitro protein accumulation.

Toxins of toxin-antitoxin (TA) systems. Nine toxins of E. coli toxin-antitoxin (TA) systems were identified as negative effectors due to mRNA degradative activities: MazF (−51%), RelE (−37%), YafQ (−16%), SymE (−24%), YoeB (−23%), YafO (−18%), YeeV (−34%), Ykfl (−26%), and YpjF (−23%). In the cell, these toxins cause cell growth arrest and eventual cell death. However, their toxicity is not exerted under normal growth conditions, for the toxins are co-transcribed with their cognate antitoxins from an operon, and they form stable complexes in the cell.

### Factors Affecting Translation

Several key factors with known or predicted involvement in each stage of translation, except tRNA aminoacylation, significantly influenced the activities of the reporters. In our survey, we identified four effectors of initiation (IF-3, IF-1, IF-2, and YciH), ten effectors of elongation (EF-Tu, EF-Ts, EF-G, EF-P, YigZ, YchF, RelA, SpoT, LepA, and RaiA), three effectors of termination (RF-1, RF-2, and RF-3), and six effectors of ribosome recycling (Frr, RF-3, EF-G, HsIR, tmRNA, and Pth). Two of these factors (EF-G and RF-3) presumably had roles in multiple phases of the in vitro translation process.

### Factors Affecting Transcription and Translation

We identified six factors that were potentially involved in enhancing the efficiency of both transcription and translation in our system. This set of positive effectors included members of the cold-shock protein A (CspA) family: CspA (+63%), CspB (+24%), CspC (+21%), CspE (+30%), CspF (+18%), and CspG (+12%), which enhanced total CAT yields.

### Factors Affecting Protein Folding

The first-round expression of several molecular chaperones and chaperone-like products significantly affected the specific activity of CAT and several other E. coli enzymes. This set of thirty-two effectors included a wide variety of proteins, such as the traditional folding helpers and catalysts (heat-shock proteins (Hsps), small heat-shock proteins (sHsps), and disulfide bond (Dsb) isomerases and oxidoreductases), which we expected to identify; as well as a number of unanticipated factors which either possess secondary chaperone-like functions or catalyze activities causing environmental changes that affect protein folding. The effectors were classified as follows: (1) Proteins of the Hsp10 and Hsp600 families (2 effectors); (2) Proteins and co-chaperone proteins of the Hsp70 family (9 effectors); (3) Proteins of the Hsp31. Hsp90, and Hsp100 families (5 effectors); (4) Small heat-shock proteins (sHsps) (2 effectors); (5) Periplasmic chaperones (1 effector); (6) Proteins that possess lesser known or secondary chaperone-like activities (7 effectors); (7) Proteins of the disulfide bond (Dsb) formation family (3 effectors); and (8) Predicted molecular chaperones (3 effectors).

TABLE 8
Effects of several positive effectors on protein specific activity. The first-round cell-free expression of several positive effectors significantly
enhanced the total enzymatic activities of the survey reporter proteins. Here we show that these effectors increase the specific activities
of several E. coli enzymes. The soluble yields were not affected. Results are the average of n = 3 experiments.

| PROTEIN | | | GroEL | | TrxA | | LepA | | YciM | | Gor | | DjlA | |
|---|---|---|---|---|---|---|---|---|---|---|---|---|---|---|
| wild-type | | | | % in- | | % in- | | % in- | | % in- | | % in- | | % in- |
| specific | | | | crease | | crease | | crease | | crease | | crease | | crease |
| activity * | Baseline | | | in | | in | | in | | in | | in | | in |
| U/mg | soluble | active * | active * | active | active * | active | active * | active | active * | active | active * | active | active * | active |
| (reference) | (μg/mL) | (%) | (%) | yields | (%) | yields | (%) | yields | (%) | yields | (%) | yields | (%) | yields |
| UMP kinase | 1179.2 ± 92.3 | 52 ± 10 | 83 ± 14 | 60 | 73 ± 14 | 40 | 71 ± 10 | 37 | 64 ± 14 | 23 | 68 ± 4 | 31 | 59 ± 2 | 13 |
| 128 U/mg | | | | | | | | | | | | | | |
| (Bucurenci | | | | | | | | | | | | | | |
| et al., 1998) | | | | | | | | | | | | | | |
| β-Lactamase | 237.3 ± 39.9 | 8 ± 1 | 13 ± 1 | 63 | 12 ± 1 | 50 | 11 ± 2 | 38 | 10 ± 2 | 25 | 10 ± 1 | 25 | 9 ± 1 | 13 |
| 1928 U/mg | | | | | | | | | | | | | | |
| (McCarthy | | | | | | | | | | | | | | |
| et al., 1998) | | | | | | | | | | | | | | |
| Thiorodoxin | 1380.3 ± 95.2 | 83 ± 5 | 100 ± 15 | 20 | 100 ± 7 | 20 | 100 ± 21 | 20 | 100 ± 4 | 20 | 100 ± 9 | 20 | 95 ± 5 | 14 |
| reductase 39 | | | | | | | | | | | | | | |
| U/mg (Knapp | | | | | | | | | | | | | | |
| and Swartz, | | | | | | | | | | | | | | |
| 2004) | | | | | | | | | | | | | | |
| Cytidine | 1289.9 ± 98.2 | 68 ± 14 | 100 ± 25 | 47 | 100 ± 21 | 47 | 87 ± 6 | 28 | 86 ± 11 | 26 | 88 ± 4 | 29 | 75 ± 6 | 10 |
| deaminase | | | | | | | | | | | | | | |
| 250 U/mg | | | | | | | | | | | | | | |
| (Yang et al., | | | | | | | | | | | | | | |
| 1994) | | | | | | | | | | | | | | |
| Malate dehy- | 1021.8 ± 87.7 | 51 ± 2 | 82 ± 21 | 61 | 76 ± 3 | 49 | 63 ± 5 | 24 | 61 ± 13 | 20 | 66 ± 6 | 29 | 57 ± 2 | 12 |
| drogenase | | | | | | | | | | | | | | |
| 700 U/mg | | | | | | | | | | | | | | |
| (Breiter, | | | | | | | | | | | | | | |
| et al. 1994) | | | | | | | | | | | | | | |
| Glucose | 468.2 ± 36.6 | 62 ± 3 | 96 ± 24 | 55 | 85 ± 4 | 37 | 84 ± 5 | 35 | 82 ± 7 | 32 | 77 ± 2 | 24 | 69 ± 5 | 11 |
| 6-phosphate | | | | | | | | | | | | | | |
| dehydro- | | | | | | | | | | | | | | |
| genase | | | | | | | | | | | | | | |
| 104 U/mg | | | | | | | | | | | | | | |
| (Banerjea | | | | | | | | | | | | | | |
| and Frankel, | | | | | | | | | | | | | | |
| 1972) | | | | | | | | | | | | | | |
| Chloram- | 1129.7 ± 32.9 | 93 ± 1 | 98 ± 27 | 5 | 96 ± 14 | 3 | 97 ± 11 | 4 | 97 ± 18 | 4 | 98 ± 21 | 5 | 98 ± 17 | 5 |
| phenicol | | | | | | | | | | | | | | |
| acetyltrans- | | | | | | | | | | | | | | |
| ferase | | | | | | | | | | | | | | |
| 125 U/mg | | | | | | | | | | | | | | |
| (Shaw, 1975) | | | | | | | | | | | | | | |

^{a} A unit is defined as micromoles of substrate catalyzed per minute.
* Determination of active fraction is based on published specific activities and on soluble accumulated protein.

### Factors Affecting Protein Stability

We identified four enzymes that are directly involved in protein degradation and amino acid recycling as negative effectors in the survey: PepN, YpdF, DegQ, and PtrA. PepN and YpdF negatively affected the accumulated activities of the three reporters similarly and reduced total CAT production by 28 and 24%, respectively; whereas, DegQ and PtrA influenced only β-lactamase and decreased its accumulated activity by 12 and 11%, respectively.

Factors with Unexplained Influences

Although extensive studies (e.g., analyses of amino acid, nucleotide, and central metabolite levels and thorough literature searches) were conducted to elucidate the in vitro activities of the identified effectors of CFPS, 8.6% of the observed effects (12 out of 139) remained unexplained. For nine of these unexplained effects, the known functions of the effectors are important for in vive metabolism and protein production, but their relevance and/or effects in the CFPS system were not clear. The effectors classified as such were positive effectors Map (+17%), AsnA (+18%), AsnB (+24%), DeaA (+29%), XthA (+34%), and UidA (+43%), which enhanced total CAT production; positive effector Ffh improved the specific activity of CAT by 37%; and negative effectors FolA and AceF reduced the accumulation of total CAT by 18 and 45%, respectively.

In this work, we conducted a genome-wide functional genomic analysis of E. coli using a sequential CFPS platform. The first round of expression was directed by linear DNA ETs and essentially created an array of cell extracts individually enriched with a single analyte. This round was terminated by the selective degradation of the linear templates, and a second round of CFPS was initiated by the addition of a plasmid ET for the chosen reporter enzyme. Changes from baseline accumulated reporter activity were calculated to identify gene products that influenced the cell-free metabolism. We identified 79 positive and 60 negative effectors with vastly divergent metabolic roles, including involvement in energy supply; mRNA synthesis; RNA, nucleotide, and amino acid stability; and translation initiation and elongation rates as well as protein folding, activation, and stability. By performing a variety of biochemical assays to elucidate the in vitro activities of the effectors, we resolved that 91.4% of the observations were consistent with the in vivo metabolic role(s) of the effectors; the remaining 8.6% remain unexplained.

These results demonstrate the utility of this sequential CFPS platform for functional genomics. By using such a complex assay system, which included the integrated processes of transcription, translation, and protein folding and the central catabolic pathways of glycolysis, the TCA cycle, and oxidative phosphorylation, we were able to examine the E. coli metabolome and study protein function in the context of this intricate metabolic network, as well as gain insights into metabolism after energy depletion. The observations from our genome-wide survey provided support for the inferred activities of ten proteins (YdaM, YddV, YneF. YhcM, EutP, YjdA, YchF, YciM, YgeG, and YcaL) and suggested new functions for twelve proteins (Map, AsnA, AsnB, DeoA, XthA, UidA, FtsZ, FtsW, and FtsL).

### Example 3

### Improvement of Cell-Free Protein Synthesis Guided by Genome-Wide Functional Genomic Analysis

The information from the genome-wide survey was used to guide modifications of the CFPS system in order to improve the productivity and duration of in vitro protein synthesis, as well as the efficiency of protein folding. First, we produced fifteen of the positive effectors and supplemented the expression reactions with various combinations of the effectors in order to identify cooperative interactions that further enhance system performance. Next, we constructed and evaluated four mutant E. coli strains with chromosomal deletions in non-essential genes that encode negative effectors identified by the genomic survey.

Our improved in vitro protein expression system includes the following modifications: (1) enhancement of energy generation, translation initiation and elongation, and protein folding via supplementation of eight purified positive effectors; (2) stabilization of reaction pH by adding 90 mM Bis-Tris, pH 7.2; (3) replenishment of limiting substrates (amino acids, UTP, and T7 RNA polymerase) by batch feeding during the incubation period; and (4) stabilization of mRNA transcripts by deleting rnb, which encodes ribonuclease II, from the genome of the source E: coli strain used for cell extract preparation. With this new system, the total, soluble, and active yields of the several diverse proteins were enhanced by 300 to 400%.

### Materials and Methods

Construction of expression plasmids. Plasmid templates were constructed for the in vivo expression and native (untagged) purification of small heat-shock proteins (sHsps) IbpA and IbpB. Expression plasmids were also constructed for the in vivo expression and His-tag purification of the following positive effectors: acetate kinase (AckA), FMN reductase (Fre), glutamate dehydrogenase (GdhA), nucleoside diphosphate kinase (Ndk), cytidine deaminase (Cdd), elongation factor 4 (LepA), elongation factor Tu (EF-Tu), 10-kDa chaperonin (GroES), 60-kDa chaperonin (GroEL), and Hsp31 (HchA). Standard procedures were used to subcone each gene into the expression vector pY71L, placing the coding region between the T7 promoter and T7 terminator. Each gene was amplified by polymerase chain reaction (PCR) using purified E. coli A19 genomic DNA as the template, which was prepared using DNeasye Blood and Tissue kit (Qiagen Ltd., Valencia, Calif.), and a pair of gene-specific oligonucleotide primers. Each sense (forward) primer included the NdeI restriction site for subsequent fragment digestion and ligation with vector, whereas each antisense (reverse) primer included the SphI site. Additionally, to obtain effectors carrying a hexahistidine tag at the C terminus, the representative antisense primers were extended at the 3′ terminus with the 6× histidine tag sequence. Each gene was amplified in a 50-μL PCR reaction containing 2.5 U Accuprime Pfx DNAP, 1 μM each of the sense and antisense primers, 2 μg/mL genomic DNA, 300 μM dNTPs and MgSO₄ to a final concentration of 1.3 mM. The targets were amplified in parallel beginning with a single incubation at 95° C. for 2 min, followed by 25 cycles of 95° C. for 15 sec, 60° C. for 30 sec, and 68° C. for 5 min. The PCR products were purified using QIAquick™ PCR purification kit (Qiagen Ltd. Valencia, Calif.) according to the manufacturer's instructions. The purified products and expression vector were subjected to NdeI/SphI digestion and subsequent separation in a 1.2% (w/v) agarose gel. A gel extraction kit (Qiagen Ltd., Valencia, Calif.) was used to recover the desired digested products. The double-stranded DNA (dsDNA) products were eluted from the capture column using 50 μL of water, and the concentration and purity of the digested products were determined by gel analysis and by measuring the absorbance at 260 and 280 nm.

In vivo expression of CFPS effectors. Each expression plasmid encoding a positive CFPS effector was used to transform E. coli BL21 (DE3) cells for T7 RNA polymerase-directed expression of the target protein. The transformed cell strains were grown in 0.5 L of 2YT broth (Teknova, Hollister, Calif.) containing 20 μg/mL kanamycin in 2 L Corning® Erlenmeyer baffled flasks (Sigma-Aldrich, St. Louis, Mo.). Cells were grown at 37° C. with agitation set at 280 rpm. At A₆₀₀≦0.5, production of the proteins was induced by the addition of 1 mM isopropyl-(3-D-thiogalactopyranoside (IPTG), and the cultures were agitated at 280 rpm and 37° C. for an additional 4 hr. The cells were harvested by centrifugation at 8,000×g and 4° C. for 15 min. The cells expressing the sHsps (IbpA and IbpB) were resuspended in 2 mL of 20 mM sodium phosphate (NaH₂PO₄), pH 7.0 per gram of wet cell mass and disrupted by a single pass through a high-pressure homogenizer (Emulsiflex C50, Avestin, Ottawa, Canada) at 20,000 psig. The insoluble fraction was sedimented by centrifugation at 12,000×g and 4° C. for 20 min. The supernatant was removed, and the pellet, which contained the expressed sHsps, was resuspended in 1 mL of 20 mM sodium phosphate (NaH₂PO₄), pH 7.0 per gram of wet cell mass. The pellet resuspensions were used for protein purification.

The cells expressing the C-terminal His-tagged effectors were chemically lysed using BugBuster® Protein Extraction Reagent (Novagen, Gibbstown, N.J.) according to the manufacturer's instructions. The cell pellets were resuspended in 5 mL of BugBuster® reagent diluted with the respective protein-specific binding buffer per gram of wet cell mass. Benzonase® nuclease (Novagen, Gibbstown, N.J.) at 25 units per 1 mL of BugBuster® reagent, rLysozyme™ (Novagen, Gibbstown, N.J.) at 1000 units per 1 mL of BugBuster® reagent, and EDTA-free protease inhibitor cocktail (Roche Applied Sciences, Indianapolis, Ind.) were also added to the cell resuspensions, which were then incubated at room temperature for 20 min on a rotating mixer. The insoluble fractions were sedimented by centrifugation at 12,000×g and 40° C. for 20 min. The supernatants were transferred to fresh Falcon tubes and were saved for His-tag protein purification. For the overexpression of Ndk and Cdd, the respective effectors were found in the insoluble fraction. In these cases, the cell pellet was washed in 5 mL of BugBuster® reagent diluted with the protein-specific binding buffer per gram of the initial wet cell mass. The inclusion bodies pellet was collected by centrifugation at 12,000×g and 4° C. for 15 min and was then resuspended in 5 mL of a 1:10 diluted BugBuster® reagent solution containing the effector-specific binding buffer and 6 M urea per gram of initial wet cell mass. The resuspension was incubated at 4° C. for 1 hr on a rotating mixer and was clarified by centrifugation at 12,000×g and 4° C. for 15 min. The supernatant was transferred to a fresh Falcon and was used for His-tag protein purification. The expression constructs isolated from cultures of individual transformants were screened by NdeI/SphI digestion and further verified by DNA sequencing (Pan and Nucleic Acid Facilities, Stanford University, Stanford, Calif.).

Purification of His-tagged positive effectors. The purification protocol (i.e., binding/wash and elution buffers) was optimized for each His-tagged effector using His SpinTrap™ columns (GE Healthcare Bio286 Sciences, Piscataway, N.J.) according to the manufacturer's instructions. Purification buffers containing 20 mM sodium phosphate (pH 7.4), 0.5 M NaCl, and various imidazole concentrations (5, 10, 20, 30, 50, 100, 150, 250, and 500 mM) were prepared, and the His SpinTrap™ columns were equilibrated with 600 μL of the 5 mM imidazole buffer. The unpurified effector samples were applied to the columns, which were then washed, and His-tagged proteins were eluted in a stepwise manner with 200 μL of the elution buffer containing the next higher imidazole concentration to the columns; the elution fractions were collected from each step. The purified proteins were stored in 10 mM potassium phosphate (pH 7.2) with 20% (v/v) sucrose. The protein solutions were flash-frozen in liquid nitrogen and stored at −20° C. until use.

SDS-PAGE. The purified protein samples were analyzed by reducing SDS-PAGE. NuPAGE® Novex precast gels and reagents were purchased from Invitrogen (Carlsbad, Calif.). Samples 30 μg/mL) were denatured for 10 min at 70° C. in loading buffer (1×LDS running buffer and 50 mM DTT). The samples were loaded onto a 10% (w/v) Bis-Tris precast gel with SeeBlue® molecular weight protein standard and electrophoresed in MES/SDS running buffer containing NuPAGE® antioxidant. SimplyBlue® SafeStain was used to stain and fix the gels according to the manufacturer's recommendations. The gels were dried using a gel dryer (Bio-Rad, Hercules, Calif.).

CFPS with supplementation of positive effectors. The biological activities of the purified positive effectors were qualitatively assessed by supplementing standard CFPS reactions with the effectors and comparing their effects on the cell-free production and activation of GFP against those observed in the sequential CFPS experiments and in CFPS reactions containing the respective commercially purified enzymes. The following enzymes were purchased to validate the activities of the purified effectors: AckA and Ndk from Sigma-Aldrich (St. Louis, Mo.); GroEL and GroES from Enzo Life Sciences (Plymouth Meeting, Pa.); and Fre from NovoCIB (Lyon, France). The purified effectors and commercially purchased enzymes were added to batch CFPS reactions at the beginning of the incubation period to a final concentration representing their respective yields after a 40-min round of cell-free expression. The sucrose from the purified effector samples was removed immediately before use by using centrifugal devices with 3-kDa molecular-weight cut-off (MWCO) membranes (Pall Corporation, New York. USA) according to the manufacturer's instructions. The sucrose was removed to prevent any potential CFPS inhibition. Total GFP accumulation and specific activity were determined as described above.

Enzymatic activity assays. All spectrophotometric and fluorescence measurements were conducted using a SpectraMax® 190 microplate spectrophotometer (Molecular Devices, Sunnyvale, Calif.) and Mithras LB 940 multimode plate reader (Berthold Technologies, Oak Ridge, Tenn.), respectively. Enzyme-specific activities were measured without purification and corrected for any detectable background activity by comparing to an equivalent volume of a CFPS reaction conducted without a DNA template.

Creation of single-gene deletion strains. The four single-gene deletion (pnp, rnb, raiA, and mazG) mutant strains were constructed with a one-step inactivation method using the phage λ Red recombinase system. Briefly, gene-specific PCR primers, each with a 50-nucleotide (nt) homologous region to one end of the gene of interest, were used to amplify the kanamycin (kan) cassette that contains FLP recognition target sites from the template plasmid pKD13 (from Coli Genetic Stock Center (CGSC) #7633). The sense (FWD) primers also include 20-nt 3′ ends for priming upstream (P1, underlined) of the pKD13 plasmid; while the antisense (REV) primers also include 20-nt 3′ ends for priming downstream (P2, underlined) of the pKD13 plasmid. Each PCR product was purified and recombined into an E. coli strain expressing the λ Red recombination system (CGSC #7739). A P1 phage lysate from these bacteria was transduced into strain KC6, a derivative of E. coli strain A19. Kanamycin resistant colonies were isolated, and these bacteria were transformed with a curable helper plasmid that encodes the FLP recombinase to eliminate the ken gene. All of the modified strains, except for IA1 (KC6Δpnp), grew similarly to KC6 on defined media with a growth rate of 1.2 hr-1. The IA1 strain had a slower growth rate of 0.9 hr-1.

Culture conditions and S30 cell extract preparation. The KC6 and mutant strains were grown to ˜4-5 OD600 in 1 L of defined media (with glucose as the carbon source) in 2 L Tunair shake flasks (Sigma-Aldrich, St. Louis, Mo.) as described elsewhere. Briefly, the cells were grown at 37° C. with shaking at 280 rpm and were harvested during mid- to late-logarithmic growth by centrifugation at 6,000×g and 4° C. for 30 min. The pellet was washed at 4° C. by resuspension in 40 mL of cold S30 buffer (10 mM Tris acetate (pH 8.2), 14 mM magnesium acetate, 60 mM potassium acetate, and 1 mM DTT) and centrifuged again; this wash step was repeated twice. The resulting cell paste was stored at −80° C. until it was processed into S30 cell extract. The frozen cell paste was thawed in 1 mL of S30 buffer per gram of wet cell mass and resuspended to homogeneity. The cells were disrupted by a single pass through a high-pressure homogenizer (Emulsiflex C50, Avestin, Ottawa, Canada) at 20,000 psig. The lysate was clarified by two rounds of centrifugation, each at 30,000×g and 4° C. for 30 min; the pellet was discarded after each spin. The ribosome run-off and selective destruction of endogenous mRNAs was accomplished by incubating the supernatant on a rotary shaker (120 rpm) at 37PC for 80 min. Three batches of crude S30 cell extract were made from each E. coli strain and were flash-frozen and stored at −80° C. pH was measured during cell-free reactions using a micro-pH probe (Model 2979810BN, Orion, Beverly, Mass.). pH standards (7.0 and 4.0) were warmed to 37° C. prior to calibration. The tip was placed in the 15-μL reaction volume in the well of the microtiter plate. Data was collected for the first 90 min of the CFPS reaction incubation period.

In vitro transcription. Radiolabeled CAT-mRNA transcript was obtained by the in vitro transcription of the pK7CAT plasmid with T7 RNA polymerase. The reaction mixture (50 μL) contained 50 mM Tris-HCl (pH 7.5), 15 mM magnesium chloride, 5 mM dithiothreitol, 2 mM spermidine, 2 mM each of ATP, GTP, CTP, and UTP, 10 μM ([³H]-UTP (Amersham GE Biosciences, Piscataway, N.J.), 1 mg/mL T7 RNA polymerase, 1 U/μL RNaseOUT RNase inhibitor (Invitrogen, Carlsbad, Calif.), and 100 μg/mL template DNA. The mixture was incubated in a humidified incubator at 37° C. for 3 hr and treated with 10 U RNase-free DNase I for 15 min at room temperature. The mRNA transcript was isolated by Trizol®/chloroform extraction according to manufacturer's instructions (Invitrogen), followed by isopropanol precipitation and ethanol wash. The pellet was immediately resuspended in RNase-free water, and the mRNA concentration and purity were measured by UV-spectroscopy using the Qubit™ fluorometer (Invitrogen). The integrity of the mRNA transcripts was analyzed by denaturing electrophoresis on 6% polyacrylamide TBE-Urea gels according to manufacturer's instructions (Invitrogen), followed by ethidium bromide staining.

mRNA decay assay. The effect of the deletion of pnp and rnb from the KC6 E. coli strain on mRNA stability was analyzed by incubating in vitro transcribed-[3H]-UTP-labeled CAT mRNA transcripts in CFPS reaction mixtures using the respective S30 cell extracts and monitoring the degree of transcript degradation. The mRNA concentration and stability in the CFPS reaction mixtures were analyzed by measuring TCA-insoluble radioactivity using a liquid scintillation counter (Wallac 1450 Microbeta LSC, Perkin Elmer) and performing denaturing electrophoresis on 6% polyacrylamide TBE-Urea gels according to manufacturer's instructions (Invitrogen, Carlsbad, Calif.).

To improve the CFPS system, we selected fifteen positive effectors of cell-free protein expression and/or folding to be produced and supplemented into the CFPS reactions. These effectors were identified in our functional genomic analysis and represented proteins with vastly divergent metabolic roles, including involvement in energy supply; nucleotide and mRNA stability; and translation initiation and elongation rates as well as protein folding. The selected proteins were categorized as follows: (1) Factors affecting the energy (ATP and GTP) supply (4 effectors); (2) Factors affecting the supply of nucleotides (NTPs) for transcription (1 effector); (3) Factors affecting translation (5 effectors); and (4) Factors affecting protein folding (5 effectors).

We selected four proteins that enhanced the ATP and GTP supply in our CFPS system glutamate dehydrogenase (GdhA), FMN reductase (Fre), acetate kinase (AckA), and nucleoside diphosphate kinase (Ndk). In our in vitro expression system, natural metabolism (i.e., glycolysis, the TCA cycle, and oxidative phosphorylation) was activated, and glutamate was used as the primary energy source. Glutamate was directed into the TCA cycle to form α-ketoglutarate by endogenous GdhA and to produce reducing equivalents. GdhA was selected for purification in order to increase the entry of glutamate into the TCA cycle and the production rate of reducing agents. These reducing equivalents from the glutamate catabolism then fuel oxidative phosphorylation with oxygen serving as the final electron acceptor, providing a stable supply of ATP that is coupled to combined transcription and translation followed by protein folding.

TABLE 9A and 9B
Table 4. Positive effectors of CFPS that were purified and supplemented into the in vitro expression reactions.
4A. Positive effectors that enhance expression of reporter proteins in CFPS reactions

| | | | | CFPS yield | Average % change |
|---|---|---|---|---|---|
| | Monomer | Multimeric | | of effector | from baseline |
| Effector Protein | MW (kDa) | state | Metabolic role | (μM) | yields of reporters |
| Acetate kinase AckA * | 43.3 | monomer | Energy | 6.4 ± 0.4 | 64.3 ± 7.0 |
| FMN reductase Fre * | 26.2 | monomer | Energy | 5.2 ± 0.3 | 37.3 ± 4.1 |
| Glutamate dehydrogenase GdhA | 48.6 | homohexamer | Energy | 0.8 ± 0.1 | 25.8 ± 2.9 |
| Nucleoside diphosphate kinase * | 15.6 | homotetramer | Energy | 2.4 ± 0.5 | 52.6 ± 5.8 |
| Cytidine deaminase Cdd | 31.5 | monodimer | Transcription | 5.5 ± 0.8 | 57.4 ± 6.3 |
| Elongation factor EF-Tu | 43.3 | monomer | Translation | 4.1 ± 0.2 | 48.9 ± 5.4 |
| Initiation factor IF-1 | 8.3 | monomer | Translation | 20.7 ± 0.8 | 12.2 ± 1.3 |
| Initiation factor IF-2 | 97.4 | monomer | Translation | 1.6 ± 0.1 | 14.7 ± 1.7 |
| Initiation factor IF-3 | 20.6 | monomer | Translation | 12.2 ± 0.8 | 8.5 ± 1.1 |
| IF-1 + IF-2 + IF-3 | — | — | — | — | 49.2 ± 5.4 |
| sHsp IbpA | 15.8 | monomer | Folding/translation | 10.4 ± 1.5 | 32.1 ± 3.5 |
| sHsp IbpB | 15.1 | homo-40-mer | Folding/translation | 0.2 ± 0.0 | 44.4 ± 4.8 |

4B. Positive effectors that enhance specific activity of reporter proteins in CFPS reactions

| | | | | CFPS | | % increase in | |
|---|---|---|---|---|---|---|---|
| | | | | yield of | % increase in | active | % increase in |
| | Monomer | Multimeric | | effector | active CAT | β-lactamase | active GFP |
| Effector Protein | MW (kDa) | state | Metabolic role | (μM) | yields | yields | yields |
| Elongation factor 4 LepA | 66.6 | monomer | Translation | 1.3 ± 0.1 | 4.4 ± 0.4 | 86.4 ± 7.6 | 8.9 ± 1.1 |
| 60-kDa chaperonin GroEL * | 57.3 | homo-14-mer | Folding | 0.1 ± 0.2 | 5.4 ± 0.6 | 111.3 ± 10.1 | 11.0 ± 0.8 |
| 10-kDa chaperonin GroES* | 10.4 | homoheptamer | Folding | 3.6 ± 2.1 | 4.0 ± 0.5 | 63.2 ± 7.2 | 6.7 ± 0.4 |
| GroEL + GroES | — | — | — | — | 7.1 ± 0.6 | 184.7 ± 12.2 | 12.2 ± 1.0 |
| Hsp31 HchA | 31.2 | homodimer | Folding | 1.3 ± 0.2 | 4.9 ± 0.3 | 78.5 ± 6.6 | 9.1 ± 0.7 |
| sHsp IbpA | 15.8 | monomer | Folding/translation | 10.4 ± 1.5 | 4.2 ± 0.7 | 96.4 ± 9.4 | 5.6 ± 0.4 |
| sHsp IbpB | 16.1 | homo-40-mer | Folding/translation | 0.2 ± 0.0 | 5.2 ± 0.8 | 159.9 ± 11.4 | 10.1 ± 0.8 |

* activity of purified effector was compared to that of commercially purified protein
Baseline CAT specific activity: 116.3 ± 11.4 U/mg
Wild-type CAT specific activity: 125 U/mg (Shaw, 1975)
Baseline β-lactamase specific activity: 10.2 ± 0.6 U/mg
Wild-type βlactamase specific activity: 128 U/mg (Bucurences et at., 1998)
Baseline GFP specific fluorescence activity: 1038.6 ± 69.4 AU
Wild-type GFP specific fluorencence activity: 1167 ± 87.3 AU (based on purified sample provided by Cem Albayrak of the Swarta group (Stanford University, Stanford, CA))
Fifteen positive effectors of cell-free protein accumulation and/or folding were produced and added to the expression reactions in order to improve the CFPS system. The effectors were identified using a sequential CFPS method (Chapter 4, Airen, 2011) and influenced various metabolic rates, such as energy and NTP supply, translation initiation and elongation, and protein folding.
The purified effectors were initially added at concentrations representing their respective yields after a 40-min round of CFPS (i.e., terminated CFPS yield), and they exerted similar effects on the expression (A) and/or activity (B) of CAT, β-lactamase, and GFP as those observed in the functional genomic analysis (Chapter 4, Auren, 2011) and those observed in CFPS reactions supplemented with commercially purified enzymes (*). Results are the average of n = 3 experiments.

Expression and purification of positive effectors of CFPS to improve in vitro protein expression. The fifteen positive effectors were individually overexpressed in E. coli 8121 (DE3). All the proteins carried a hexahistidine tag at the C-terminus, except for IbpA and IbpB, which were expressed in their native forms. Two of the overexpressed His-tagged proteins (Ndk and Cdd) were not found in the soluble fractions of the cell lysates, but in the rapidly sedimenting fraction containing aggregated proteins and membrane vesicles. These effectors were extracted from the aggregates by dissolving them in 6 M urea, and purification was performed under denaturing conditions. The purified proteins were then refolded by performing a buffer exchange with a gel filtration matrix in order to remove the 6 M urea.

The final samples of each purified effector were approximately 95-98% pure as judged by densitometric scanning of overloaded gels. Effects of supplementing purified positive effectors into CFPS reactions The activity of each purified effector was qualitatively assessed by supplementing the standard CFPS reactions with the purified proteins and comparing the effects against those observed in the sequential CFPS experiments and/or in the reactions enriched for the respective commercially purified products.

The purified proteins were added to the expression reactions at concentrations representing their respective yields after a 40-min round of CFPS (i.e., terminated CFPS yields or initial concentrations). Each purified effector was evaluated for its ability to increase protein accumulation and/or folding by determining the percent change from baseline concentration and baseline specific activity, which were measured in batch CFPS reactions without supplementation (i.e., negative control). The effector activities were determined for three different reporter proteins: chloramphenicol acetyltransferase (CAT), β-lactamase, and green fluorescent protein (GFP). CAT, a 75-kD trimeric enzyme (25 kD monomer), is a well-characterized reporter system and a model protein often expressed in bacterial cell-free systems.

Protein yields increased in the presence of the following effectors: GdhA, AckA, Fre, Ndk, Cdd, IF-1, IF-2, IF-3, and EF-Tu; whereas, the activities of LepA, GroEL, GroES, and HchA improved the specific activities of the reporters. In the IbpA- and IbpB-enriched CFPS reactions, we observed improvements in both protein accumulation and specific activity. These results validated the activities of the purified effector products.

We then sought to optimize the amount of each effector added to the CFPS reaction in order to further improve protein synthesis and folding. Using the CFPS yields of the effectors in the genomic screen as the initial supplemented concentrations, we examined the effects of different protein concentrations on the accumulation and activity of GFP.

We supplemented the expression reactions with the purified proteins at concentrations that were half, twice, and four times those of the genomic survey CFPS yields. For all the effectors, the optimal supplemented amount was either the initial or twice the initial concentration, for these were the minimum concentrations that still provided significant improvement. In all the cases, quadrupling the initial supplemented concentration did not provide appreciable benefits. This observation strongly implies that there are multiple areas of limitation in the CFPS system and that only limited improvement can be achieved by individually addressing individual bottlenecks.

Conditions of the design of experiment (DoE) analysis. To effectively utilize the survey data for the improvement of the CFPS system, a statistical design of experiment (DoE) approach was adopted to identify combinations of the positive effectors that exhibit cooperative interactions. These combinations would potentially further enhance the performance of the in vitro protein expression system. The DoE Design-Ease® 7.1.6 software (Stat-Ease, Inc., Minneapolis, Minn.) was used to create a 2-level. 11-factor fractional factorial design (i.e., 211-6 design of resolution IV) and to statistically analyze the data. The levels referred to the amount (μM) of purified protein added to the CFPS reactions (0 μM and the optimal supplemented concentration determined for each effector), and the 11 factors were as follows: GdhA, AckA, Fre, Ndk, Cdd, (IF-1+IF-2+IF-3), LepA, (GroEL+GroES), HchA, IbpA, and IbpB. For the DoE analysis, the three initiation factors and the Hsp60/Hsp10 chaperones were combined and treated as single multi-effector factors to better represent their in vivo activities. These complexed factors, consequently, had a greater impact on the CFPS system than the individual components.

Furthermore, since translation elongation had already been shown to be a rate-limiting step in CFPS (Underwood et al., 2005), EF-Tu was added to all the expression reactions conducted for the DoE analysis and was not considered as one of the design factors. Also, the concentration of UTP added at the beginning of the reaction was increased to 2 mM in order to reduce the substrate depletion problem. The DoE design consisted of 32 combinations of the 11 protein factors to be analyzed, and each combination included 5-7 effectors, except the cases in which none or all of the factors were tested in a single reaction mixture (sets 1 and 32, respectively. Initial DoE experiments failed to identify any significant factors or interactions. In many of the effector-enriched CFPS reactions, we observed modest increases in the GFP accumulation (˜70% improvements from the baseline yields of 1153.7 μg/mL). Based on the fact that various effectors ware present in the expression reactions, we expected more substantial increases and more variation in the observed improvements. It thus seemed that factors other than those associated with catalyst or chaperone activities might be limiting product accumulation. Interestingly, most of these production-limiting reactions were supplemented with an effector set containing AckA. From this observation, we reasoned that the presumed cessation of protein accumulation may be associated with the activity of this enzyme.

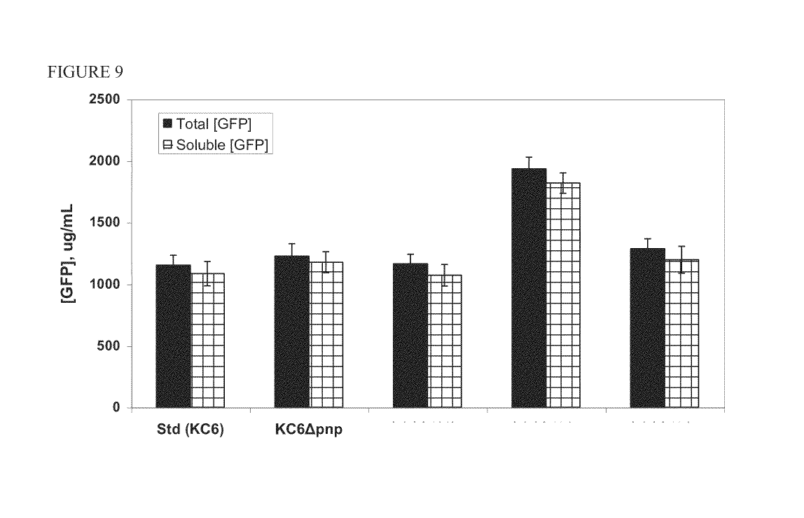

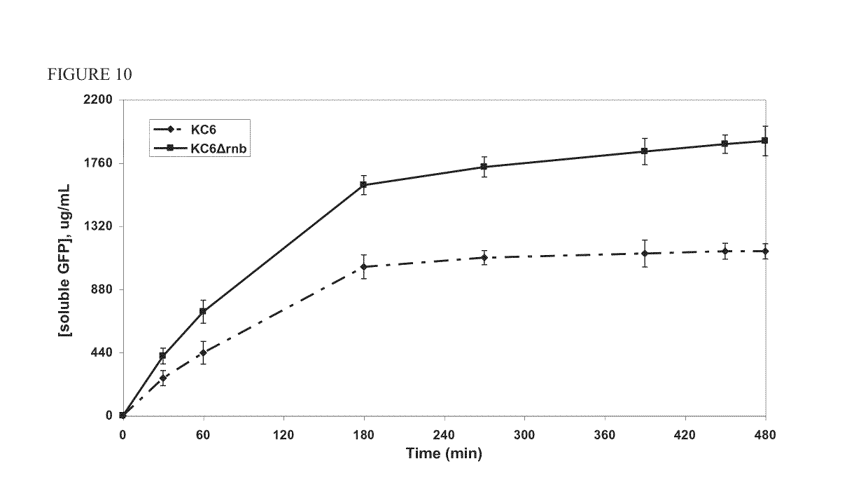

Two factors were determined as the primary reasons for termination of protein production in these CFPS reaction mixtures: significant decreases in reaction pH and depletion of multiple small molecular weight substrates. Within the first 30 min of the incubation period, the pH dropped to values known to inhibit protein synthesis, in contrast to the control (AckA-, EF-Tu-free) reactions that maintained a relatively stable pH. This substantial pH drop in the effector supplemented reactions was largely due to the increased acetate accumulation associated with the AckA-catalyzed reaction. In order to stabilize the reaction pH, 90 mM Bis-Tris (pH 7.2) (pKa ˜6.5) was added to the in vitro expression reaction mixtures. This buffer addition maintained the pH>6.8 over the course of a 5-hr CFPS reaction (FIG. 9) and, consequently, improved GFP production by ˜35% relative to the AckA-, EF-Tu-enriched reactions without pH control. The other factor associated with protein synthesis cessation was the selective depletion of essential small molecules, specifically UTP and amino acids (phenylalanine, leucine, threonine, and glycine). The concentrations of the remaining 3 nucleotides and 16 amino acids were not depleted during the course of the CFPS reaction.

In the negative control CFPS reactions and those supplemented with protein cocktails lacking Cdd, the supply of UTP depleted within the first 2 hr of the incubation period, despite the higher UTP concentration added at the beginning of the reaction.

To overcome the problem of substrate limitation, we adopted a fed-batch CFPS approach, in which the depleted small molecules were replenished every 1.5 hr over the course of a 5-hr reaction by adding 0.86 mM of UTP and 1 mM of each depleted amino acid. With this substrate replenishment in the AckA-, EF-Tu-enriched reactions, GFP yields were ˜35% higher than those in the AckA-, EF-Tu-enriched reactions without batch feedings or pH control, thus producing 2.3 mg of soluble GFP per mL.

Construction and evaluation of mutant E. coli strains carrying single deletions of non-essential genes encoding negative effectors of CFPS. We constructed four modified E. coli KC6 strains, with each containing a single deletion of one of the targeted negative-effector genes. The method of Datsenko and Wanner (2000) was used to create in-frame, markerless deletions in the KC6 chromosome, resulting in final mutant strains.

All the mutant strains, except for IA1 (KC6Apnp), grew similarly to the control strain, KC6, with growth rates of approximately 1.2 hr-1. The growth rate of the pnp deletion strain was ˜25% lower than the others, which is a trend previously observed for A pnp mutants. S30 cell extracts were prepared from the control and modified strains using the method of Yang et al. Although the growth rate of strain IA1 was slower than the other strains, it was high enough such that the integrity and productivity of the respective cell extract was not affected. The extracts were used in standard CFPS reactions producing GFP, and the protein yields obtained with the different extracts were compared. Relative to the control reactions using the KC6 extract, substantial improvements in GFP production were observed only in reactions using the extract from strain IA2 (KC6Δrnb). With this modified S30 extract, protein accumulation increased by ˜70% relative to the control CFPS reactions, yielding ˜2 mg/mL.

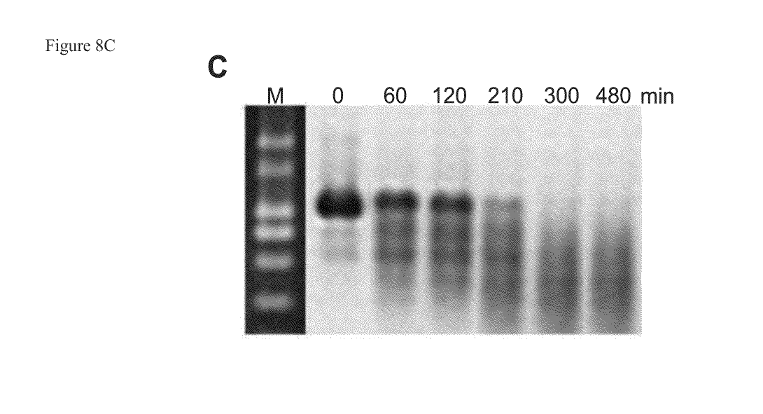

TABLE 10
FIG. 8. Experimental design for identification of positive effector combinations
enhaucing CFPS.

| | reactor | | | | | | | | | | |
|---|---|---|---|---|---|---|---|---|---|---|---|
| sat | Fre | AckA | HchA | GroEL/ES | Cdd | LepA | GdhA | IF 1-3 | IbpA | Ndk | IbpB |
| 1 | − | − | − | − | − | − | − | − | − | − | − |
| 2 | + | − | − | − | − | + | − | − | + | + | − |
| 3 | − | + | − | − | − | + | + | − | − | − | + |
| 4 | + | + | − | − | − | − | + | − | + | + | + |
| 5 | − | − | + | − | − | + | + | + | + | − | − |
| 6 | + | − | + | − | − | − | + | + | − | + | − |
| 7 | − | + | + | − | − | − | − | + | + | − | + |
| 8 | + | + | + | − | − | + | − | + | − | + | + |
| 9 | − | − | − | + | − | − | + | + | + | + | + |
| 10 | + | − | − | + | − | + | + | + | − | − | + |
| 11 | − | + | − | + | − | + | − | + | + | + | − |
| 12 | + | + | − | + | − | − | − | + | − | − | − |
| 13 | − | − | + | + | − | + | − | − | − | + | + |
| 14 | + | − | + | + | − | − | − | − | + | − | + |
| 15 | − | + | + | + | − | − | + | − | − | + | − |
| 16 | + | + | + | + | − | + | + | − | + | − | − |
| 17 | − | − | − | − | + | − | − | + | − | + | + |
| 18 | + | − | − | − | + | + | − | + | + | − | + |
| 19 | − | + | − | − | + | + | + | + | − | + | − |
| 20 | + | + | − | − | + | − | + | + | + | − | − |
| 21 | − | − | + | − | + | + | + | − | + | + | + |
| 22 | + | − | + | − | + | − | + | − | − | − | + |
| 23 | − | + | + | − | + | − | − | − | + | + | − |
| 24 | + | + | + | − | + | + | − | − | − | − | − |
| 25 | − | − | − | + | + | − | + | − | + | − | − |
| 26 | + | − | − | + | + | + | + | − | − | + | − |
| 27 | − | + | − | + | + | + | − | − | + | − | + |
| 28 | + | + | − | + | + | − | − | − | − | + | + |
| 29 | − | − | + | + | + | + | − | + | − | − | − |
| 30 | − | − | + | + | + | − | − | + | + | + | − |
| 31 | + | − | + | + | + | − | − | + | + | + | − |
| 32 | + | + | + | + | + | + | + | + | + | + | + |

The purified positive effectors were added to the CFPS reactions as their optimal concentrations. Batch reactions expressing GFP were conducted as described in the Experimental section, with one of the 32 effector cocktails added to the reaction at the beginning of the incubation period. The total and soluble yields of GFP were determined, and analysis of variance (ANOVA) was performed in order to identify the effector(s) and interactions with statistically significant effects on GFP production and solubility.

In many of the effector-enriched CFPS reactions, we observed modest increases in the GFP accumulation (˜70% improvements from the baseline yields of 1153.7 μg/ml). Based on the fact that various effectors were present in the expression reactions, we expected more substantial increases and more variation in the observed improvements. Factors other than those associated with catalyst or chaperone activities might be limiting product accumulation. Interestingly, most of these production-limiting reactions were supplemented with an effector set containing AckA.

To identify the potential non-protein-associated causes of protein synthesis arrest, we investigated the central metabolite, amino acid, and nucleotide levels, as well as the pH profile, in the reactions enriched for AckA and EF-Tu. Two factors were determined as the primary reasons for termination of protein production in these CFPS reaction mixtures: significant decreases in reaction pH and depletion of multiple small molecular weight substrates. Within the first 30 min of the incubation period, the pH dropped to values known to inhibit protein synthesis (FIG. 9), in contrast to the control (AckA-, EF-Tu-free) reactions that maintained a relatively stable pH. This substantial pH drop in the effector supplemented reactions was largely due to the increased acetate accumulation associated with the AckA-catalyzed reaction (acetyl-phosphate+ADP+H₊→acetate+ATP).

In order to stabilize the reaction pH, 90 mM Bis-Tris (pH 7.2) (pKa ˜6.5) was added to the in vitro expression reaction mixtures. This buffer addition maintained the pH>6.8 over the course of a 5-hr CFPS reaction and, consequently, improved GFP production by ˜35% relative to the AckA-, EF-Tu-enriched reactions without pH control.

The other factor associated with protein synthesis cessation was the selective depletion of essential small molecules, specifically UTP and amino acids (phenylalanine, leucine, threonine, and glycine). The concentrations of the remaining 3 nucleotides and 16 amino acids were not depleted during the course of the CFPS reaction. In the negative control CFPS reactions and those supplemented with protein cocktails lacking Cdd, the supply of UTP depleted within the first 2 hr of the incubation period, despite the higher UTP concentration added at the beginning of the reaction. The reason for this degradation still remains unknown. The UTP supply in the effector-enriched reactions depleted at rates faster than those in the negative controls, which suggests that the presence of the various effectors enhanced the rate of transcription, thus causing the quicker decrease in the UTP concentration.

These observed depletions resulted from the incorporation of the entire amino acid supply (2 mM) into the translated GFP, which has a high content of the four depleted residues (phenylalanine-15; leucine-18; threonine-15; and glycine-22). Thus, on a molar basis and taking into account the amino acid composition of our reporter protein, the concentrations of these small molecules in the reaction mixtures were sufficient for the production of −3 mg/mL of GFP, which was achieved in CFPS reactions with pH control and UTP replenishment.

To overcome the problem of substrate limitation, we adopted a fed-batch CFPS approach, in which the depleted small molecules were replenished every 1.5 hr over the course of a 5-hr reaction by adding 0.86 mM of UTP and 1 mM of each depleted amino acid. With this substrate replenishment in the AckA-, EF-Tu-enriched reactions, GFP yields were ˜35% higher than those in the AckA-, EF-Tu-enriched reactions without batch feedings or pH control, thus producing 2.3 mg of soluble GFP per ml. GFP yields were further enhanced by approximately 5% with the supplementation of additional T7 RNA polymerase (RNAP) at t_{reaction}=1.5 and 3 hr as well.

This modified CFPS system (with pH stabilization and substrate replenishment) was used for the DoE study. Analysis of variance (ANOVA) was applied in order to identify the positive effector(s) and interactions with statistically significant effects on GFP production and activity. AckA was identified as the most significant positive effector (p-value <0.0001), which was consistent with the data from our functional genomic survey of E. coli. The addition of this single factor to the modified CFPS system improved GFP yields by ˜60% over AckA-enriched standard reactions (no pH stabilization or substrate replenishment) and ˜160% over the non-supplemented (control) reactions. Although no significant interactions between any of the factors were identified, a set of 8 effectors enhanced the production of total and soluble GFP by ˜220%, relative to the non-supplemented (control) reactions, accumulating almost 4 mg/mL of soluble GFP. This effector cocktail consisted of gene products that influenced diverse metabolic areas, including energy generation (AckA); translation (IF-1, IF-2, IF-3, EF-Tu, IbpA, and IbpB); and protein folding (HchA, IbpA, and IbpB).

To further improve the performance of the in vitro protein synthesis system, nonessential genes encoding highly-negative effectors of CFPS were targeted for deletion from the source E. coli strain used for cell extract preparation, KC6. The targeted genes were pnp (encoding polynucleotide phosphorylase (PNPase)), rnb (encoding ribonuclease II (RNase II)), raiA (encoding ribosome associated inhibitor), and mazG (encoding nucleoside triphosphate pyrophosphohydrolase). Each of the respective products was identified by the genome wide survey as an inhibitor of cell-free protein accumulation.

The exoribonucleases PNPase and RNase II reduced total CAT accumulation by 33 and 46%, respectively. In vivo, PNPase and RNase II play a significant role in the decay of single-stranded mRNA by processively degrading the nucleic acid molecule in the 3′ to 5′ direction. PNPase catalyzes the phosphorolytic reaction, thus generating ribonucleoside 5′-diphosphates (RNA₍ₙ₎+phosphate→RNA₍ₙ₋₁₎+a nucleoside 5′-diphosphate); whereas, RNase II hydrolyzes mRNA to ribonucleoside 5′-monophosphates(RNA₍ₙ₎+H₂O→RNA₍ₙ₋₁₎+a nucleoside 5′-phosphate).

Since the level of gene expression is greatly affected by mRNA stability, the observed yield decreases were presumably due to the rapid degradation of the mRNA transcript in the nuclease-rich CFPS reactions. We anticipated that the deletion of the genes encoding these enzymes would increase mRNA stability and steady state levels in the expression reactions.

Another gene target for deletion was raiA, whose product is known to interfere with translation elongation by interacting with the 30S subunit of the 70S translation complex. RaiA blocks the access of aminoacyl-tRNA to the ribosomal A site. This presumed obstruction of the A site entry and subsequent inhibition of protein synthesis caused a 27% decrease in the total protein accumulation of CAT in CFPS reactions enriched for RaiA. We sought to test if the deletion of this gene from the KC6 cell strain would improve the rate of translation elongation, resulting in improved product accumulation.

We also targeted the deletion of the gene encoding the potent nucleotide hydrolase, MazG (NTP+H₂O→NMP+diphosphate). With the enrichment of CFPS reactions for this enzyme, reductions in the NTP supply were observed. These lower NTP levels presumably slowed both transcription and translation by limiting the supply of energy carriers (ATP and GTP) and precursors for RNA synthesis. These limitations caused a 35%-decrease in total protein accumulation. We expected that the absence of this enzyme in the CFPS system would decrease NTP loss, specifically that of UTP, and, consequently, enhance the levels of mRNA and duration of protein synthesis.

We constructed four modified E. coli KC6 strains, with each containing a single deletion of one of the targeted negative-effector genes. The method of Datsenko and Wanner (2000) was used to create in-frame, markerless deletions in the KC6 chromosome, resulting in final mutant strains of genotypes that are listed below.

TABLE 11
Table 3. Bacterial strains used in this study.

| E. coli | | |
|---|---|---|
| Strain | Genotype | Reference |
| A19 | Rna-19gdhA2his-95^{a}relA1spaT1 metB1 | Gesteland, 1966 |
| KC6 | A19ΔspeAΔtnaAΔsdaAΔ- | Calhoun, 2006 |
| | sdaBΔgshAΔtonAΔendAmet⁺ | |
| IA1 | KC6Δpnp | This work |
| IA2 | KC6Δrnb | This work |
| IA3 | KC6ΔraiA | This work |
| IA4 | KC6ΔmazG | This work |

^{a}The A19 strain in our laboratory has reverted to histidine prototrophy.

All the mutant strains, except for IA1 (KC6Δpnp), grew similarly to the control strain, KC6, with growth rates of approximately 1.2 hr⁻¹. The growth rate of the pnp deletion strain was ˜25% lower than the others, which is a trend previously observed for Δpnp mutants.

S30 cell extracts were prepared from the control and modified strains using the method of Yang et al. Although the growth rate of strain IA1 was slower than the other strains, it was high enough such that the integrity and productivity of the respective cell extract was not affected. The extracts were used in standard CFPS reactions producing GFP, and the protein yields obtained with the different extracts were compared. Relative to the control reactions using the KC6 extract, substantial improvements in GFP production were observed only in reactions using the extract from strain IA2 (KC6Δrnb). With this modified S30 extract, protein accumulation increased by ˜70% relative to the control CFPS reactions, yielding ˜2 mg/ml.

To investigate the reasons for these observed effects, we studied the various parameters and areas that we anticipated would be affected by each gene deletion, which included translation elongation rates and NTP and mRNA stability. In evaluating the translation elongation rates in CFPS reactions using the different cell extracts, we observed that none of the extracts from modified strains affected the rate of elongation. We had expected that the deletion of raiA would improve the translation rate by eliminating the presumed inhibition of aminoacyl-tRNA binding by RaiA. The fact that the ΔraiA mutation did not improve the CFPS performance suggests that (1) aminoacyl-tRNA binding was not a major limitation in the cell-free translation process, and/or (2) the concentrations of RaiA are not sufficient enough in the cell extract to exert an effect.

We also investigated the stability of the NTPs in the CFPS reactions using the cell extracts from the mutant E. coli strains. The concentration time courses for these substrates were very similar for all the reactions, except that GTP, ATP, and CTP levels were maintained at relatively higher constant values in the reactions using the cell extract from strain IA4 (KC6ΔmazG), as anticipated. However, the UTP stability was not affected, for the substrate supply rapidly diminished within 1 hr in all the CFPS reactions, as we have seen previously. This UTP depletion has been identified as a cause for cessation of protein synthesis in batch CFPS reactions and has been overcome with repeated batch feedings of the depleted substrate. The fact that the IA4 extract led to increased levels of the relatively stable ATP, GTP, and CTP, but not UTP, in the CFPS reactions explains the lack of protein yield improvement.

Finally, we evaluated the stability of mRNA transcripts in the expression reactions, which we believed would be affected by at least two of the gene deletions, pnp and rnb. However, improved mRNA stability was observed only in the CFPS reactions using the cell extract from strain IA2 (KC6Δrnb), which was the reason for the observed increase in protein yields. Although our functional genomic analysis and previous studies showed that the overexpression of these exonucleases in cell-free and in vivo systems caused substantial RNA degradation, it is possible that the concentration of PNPase was relatively low in the control cell extract (KC6) and, thus, was not responsible for the mRNA degradation that occurred in the control reactions. This assumption would explain why the deletion of the pnp gene from the KC6 genome did not improve the performance of the CFPS system.

All of the modifications that were guided by the genome-wide survey data were incorporated into a single in vitro expression system. These improvements consisted of the following: (1) enhancement of energy generation, translation initiation and elongation, and protein folding via supplementation of purified AckA, IF1-3, EF-Tu, IbpA, IbpB, and HchA; (2) stabilization of reaction pH by adding 90 mM Bis-Tris, pH 7.2; (3) replenishment of limiting substrates via batch feeding of amino acids (for GFP producing reactions: phenylalanine, leucine, threonine, and glycine), UTP, and T7 RNAP during the incubation period; and (4) stabilization of mRNA transcripts by deleting rnb from the genome of the source E. coli strain used for cell extract preparation (KC6), resulting in strain IA2. The specific amino acids that were fed in the CFPS reactions depended on the amino acid composition of the translated protein, and the concentrations of abundantly incorporated amino acids were replenished.

We used this improved system for the cell-free expression of five diverse proteins (GFP, CAT, β-lactamase, v-tPA, and urokinase protease). The amino acids that were replenished in the CFPS reactions expressing the different reporter proteins are as follows: (1) for GFP-glycine, leucine, phenylalanine, and threonine; (2) for CAT—glutamine, phenylalanine, and threonine; (3) for β-lactamase—asparagine, glutamine, glycine, leucine, lysine, serine, threonine, and tyrosine; (4) for v-tPA—arginine, asparagine, aspartic acid, cysteine, glutamine, glycine, leucine, lysine, serine, threonine, and tyrosine; and (5) urokinase protease—alanine, arginine, asparagine, aspartic acid, cysteine, glutamic acid, glutamine, glycine, histidine, isoleucine, leucine, lysine, proline, serine, threonine, tyrosine, and valine.

Both v-tPA and urokinase are complex proteins that require disulfide bond formation for activity, v-tPA was the third assay protein in our functional genomic survey. It is a truncated variant of the structurally complex mammalian tPA and consists of the kringle 2 and protease domains of the full-length protein, v-tPA requires a rather challenging folding process, which includes the formation of nine disulfide bonds that are necessary for activity. The urokinase protease that was expressed in our system is the 30-kDa serine protease domain of mammalian urokinase. It requires six disulfide bonds and is known to be highly dependent on disulfide isomerization. The expression of these disulfide bond-containing enzymes required additional modifications to the CFPS reactions, which included treatment of the cell extract with iodoacetamide and addition of a glutathione buffer and DsbC enzyme to the reaction mixtures.

With the improved CFPS system, the total, soluble, and active yields of the five proteins were enhanced by 300 to 400%, relative to standard (control) reactions that did not include any of the modifications. Encouragingly, despite the substantial increase in the total protein accumulation and apparent translation elongation rate (˜215% increase, from 0.37 AA/s to 1.18 AA/s, for GFP production), the fractions of soluble and active protein were similar to or higher than those in the control reactions.

Also, in the CFPS reactions expressing GFP, we observed that the duration of protein synthesis was extended to almost 7 hr using the new in vitro expression system, compared to the ˜5-hr production period in the control reactions with substrate replenishment. This observation highlights the importance of the newly enhanced protocol for cell-free protein production and further supports our conclusion that the identified metabolic processes of the in vitro metabolism are major limitations in the standard CFPS system.

TABLE 12
Table 6. Effects of the CFPS system modifications on protein production, solubility,
and specific activity.

| | Standard System | | | Improved System | | | Fold improvements | | |
|---|---|---|---|---|---|---|---|---|---|
| | Total | Soluble | Active | Total | Soluble | Active | in Yields | | |
| | (μg/mL) | (μg/mL) | (%)* | (μg/mL) | (μg/mL) | (%)* | Total | Soluble | Activity |
| Green fluorescent | 1153.7 ± 80.2 | 1084.2 ± 78.2 | 89 ± 9 | 5016.1 ± 87.6 | 4717.0 ± 77.2 | 93 ± 10 | 4.4 | 4.1 | 4.6 |
| protein (GFP) | | | | | | | | | |
| Chloramphenical | 1201.6 ± 91.1 | 1129.7 ± 78.6 | 98 ± 6 | 4489.7 ± 111.4 | 4237.8 ± 123.9 | 97 ± 11 | 3.8 | 3.7 | 3.9 |
| acetyltransferase | | | | | | | | | |
| (CAT) | | | | | | | | | |
| β-Lactamase | 263.6 ± 32.4 | 232.4 ± 28.2 | 8 ± 1 | 1239.2 ± 82.6 | 1046.8 ± 52.3 | 82 ± 6 | 4.7 | 4.5 | 15.9 |
| Urokinase | 267.6 ± 19.6 | 161.5 ± 27.1 | 42 ± 7 | 1052.9 ± 87.8 | 653.7 ± 57.6 | 61 ± 9 | 3.4 | 3.5 | 5.2 |
| vtPA | 322.3 ± 21.7 | 213.4 ± 32.7 | 53 ± 11 | 1194.4 ± 67.2 | 768.4 ± 32.5 | 74 ± 9 | 3.7 | 3.8 | 5.0 |

*Determination of active fraction is based on total soluble accumulated protein.
The total, soluble, and active yields of several proteins were significantly greater in the improved CFPS reactions (i.e., enhancement of multiple metabolic areas, pH control, substrate replenishment, and mRNA stabilization) than those in the standard reactions. Results are the average of n = 3 experiments

What is claimed is:

1. An extract of a microbial cell, wherein the extract comprises one or both of (i) increased levels of a microbial protein that increases synthetic yield in a cell-free polypeptide reaction; and (ii) decreased levels of a microbial protein that decreases synthetic yield in a cell-free polypeptide reaction; relative to the basal protein levels and synthetic activity of an extract from said microbial cell.

2. The extract of claim 1, wherein said microbial protein that decreases synthetic yield in a cell-free polypeptide reaction is ribonuclease II (rnb).

3. The extract of claim 2, wherein said microbial cell comprises an inactivated gene for rnb.

4. The extract of claim 1, comprising one or more microbial proteins that increase synthetic yield in a cell-free polypeptide reaction, selected from acetate kinase (AckA); elongation factor Tu (EF-Tu), heat shock protein 31 (HchA), small heat shock protein (IbpA); small heat shock protein (IbpB); initiation factor 1 (IF-1), initiation factor 2 (IF-2) and initiation factor 3 (IF 3).

5. The extract of claim 4, wherein said microbial protein that increases synthetic yield in a cell-free polypeptide reaction is present at greater than 2 times the basal level.

6. The extract of claim 1, wherein the microbial protein that increases synthetic yield in a cell-free polypeptide reaction is AckA.

7. The extract of claim 6, comprising increased levels of each of acetate kinase (AckA); elongation factor Tu (EF-Tu), heat shock protein 31 (HchA), small heat shock protein (IbpA); small heat shock protein (IbpB); initiation factor 1 (IF-1), initiation factor 2 (IF-2) and initiation factor 3 (IF 3).

8. The extract of claim 4, wherein said microbial cell comprises an expression construct said microbial protein that increases synthetic yield in a cell-free polypeptide reaction.

9. The extract of claim 4, wherein said microbial protein that increases synthetic yield in a cell-free polypeptide reaction is exogenously synthesized.

10. The extract of claim 1, wherein said microbial cell is E. coli.

11. The extract of claim 10, wherein said extract is an S30 extract.

12. A reaction mixture suitable for cell-free polypeptide synthesis comprising an extract according to claims 1.

13. The reaction mixture of claim 12, further comprising a pH buffer.

14. A method of cell-free polypeptide synthesis, the method comprising:
incubating a polynucleotide encoding a polypeptide of interest in a reaction mixture comprising an extract according to claim 1 for a period of time sufficient to synthesize said polypeptide.

15. The method of claim 14, wherein said reaction mixture comprises a pH buffer.

16. The method of claim 14, wherein said incubating is performed as a batch reaction.

17. The method of claim 16, wherein said reaction mixture is supplemented over time with substrate monomers that are limiting for the polypeptide of interest for production.

18. The method of claim 17, wherein said substrate monomers are amino acids.

19. A method of enhancing synthesis of a protein in a cell free protein synthesis (CFPS) reaction, the method comprising:
surveying the genome of an organism that is a source for biological extracts used in the CFPS by sequential expression to identify candidate effector genes;
expressing a target protein in a CFPS reaction combining an addition or deletion of two or more of said candidate effector genes from multiple different metabolic systems;
identifying a combination of said two or more of said candidate effector genes from multiple different metabolic systems that provides for enhanced expression of the target protein; and
adjusting one or more of substrate concentration and physical chemical environment to fully activate the CFPS reaction and prolong its synthetic life.


SEQUENCE LISTING (7 sequences)

SEQ ID NO: 1 - DNA, 26 bp, E. Coli
gtttaacttaagaaggagatatacat

SEQ ID NO: 2 - DNA, 23 bp, E. Coli
cagcggtggcagcagccaactca

SEQ ID NO: 3 - DNA, 58 bp, E. Coli
atgcaggtcatccgaggggttaacgagttcgcggccgcttaggcaccccaggctttac

SEQ ID NO: 4 - DNA, 64 bp, E. Coli
catatgtatatctccttcttaaagttaaacaaaatgatctctagatcgaaaccgttgtggtctc

SEQ ID NO: 5 - DNA, 23 bp, E. Coli
tgagttggctgctgccaccgctg

SEQ ID NO: 6 - DNA, 60 bp, E. Coli
atgcaggtcatccgaggggttaacgagttcgacgagcgtcagcttgcatgccctgcagct

SEQ ID NO: 7 - DNA, 21 bp, E. Coli
atgcaggtcatccgaggggtt